(19) United States
(12) Patent Application Publication
(10) Pub. No.: US 2005/0170352 A1
(43) Pub. Date: Aug. 4, 2005

(54) Use of biomarkers to detect breast cancer

(75) Inventors: Daniel W Chan, Clarksville, MD (US); Zhen Zhang, Dayton, MD (US); Jinong Li, Ellicott City, MD (US)

Correspondence Address: EDWARDS & ANGELL, LLP, P.O. BOX 55874, BOSTON, MA, 02205, US

(73) Assignee: Johns Hopkins University, Baltimore, MD (US)

(21) Appl. No.: 10/506,725

(22) PCT Filed: Mar. 6, 2003
(86) PCT No.: PCT/US03/06850

Related U.S. Application Data
(60) Provisional application No. 60/362,473, filed on Mar. 6, 2002.

(51) Int. Cl. C12Q 1/68; G01N 33/574

(52) U.S. Cl. 435/6; 435/7.23

(57) ABSTRACT

The present invention relates to a method for identification of tmor biomarkers markers for breast cancer, with high specificity and sensitivity. Preferred methods of the invention include qualifying breast cancer status in a subject comprising: (a) measuring at least one of the disclosed biomarkers in a sample from the subject; and (b) correlating the measurement with breast cancer status. The invention further relates to kits for qualifying breast cancer status in a subject.

The present application claims the benefit of U.S. provisional application No. 60/362,473 filed Mar. 6, 2002, and which is incorporated herein by reference in its entirety.

### FIELD OF THE INVENTION

The invention provides for high specificity and sensitivity in the detection and identification of biomarkers, important for the diagnosis, prognosis and identification of tumor stage progression in breast cancer. The plasma protein profile in breast cancer patients are distinguished from non-neoplastic individuals using biochip arrays and SELDI analysis. This technique provides a simple yet sensitive approach to diagnose breast cancer using plasma samples.

### BACKGROUND OF THE INVENTION

Based on the National Cancer Institute (NGI) incidence and National Center for Health Statistics (NCHS) mortality data, the American Cancer Society estimated that breast cancer would be the most commonly diagnosed cancer among women in 2002 in the United States. It is expected to account for 31 percent (203,500) of all new cancer cases among women and 39,600 will die from this disease. Jemal A, Thomas A, Murray T, Thun M. Cancer statistics, 2002. CA Cancer J Clin. 2002;52:23-47. Presymptomatic screening to detect early-stage cancer while it is still respectable with potential for cure can greatly reduce breast cancer related mortality. Unfortunately, only about 50% of the breast cancers are localized at the time of diagnosis. National Cancer Institute. Cancer Net PDQ Cancer Information Summaries. Monographs on “Screening for breast cancer.” http://cancer net.nci.nih.gov/pdq.html (Updated January 2001). Despite the availability and recommended use of mammography for women age 40 and older as a routine screening method, its effectiveness on reducing overall population mortality from breast cancer is still being investigated. K. Antman et al., JAMA. 1999;281:1470-2. Currently, serum tumor markers that have been investigated for use in breast cancer detection still lack the adequate sensitivity and specificity to be applicable in detecting early-stage carcinoma in a large population. The FDA approved tumor markers such as CA15.3 and CA27.29, are only recommended for monitoring therapy of advanced breast cancer or recurrence. D. W. Chan et al., J Clin. Oncology. 1997;15:2322-2328. New biomarkers that could be used individually or in combination with an existing modality for cost-effective screening of breast cancer are still urgently needed.

### SUMMARY OF THE INVENTION

The present invention provides, for the first time, novel protein markers that are differentially present in the samples of tumors at different clinical stages. The measurement of these markers, alone or in combination, in patient samples provides information that diagnostician can correlate with a probable diagnosis of different clinical stages of human cancer. This is especially important when trying to identify biomarkers in pre-invasive tumors, whereby, such a diagnosis would be life saving.

Protein markers of the invention can be characterized in one or more of several respects.

In particular, in one aspect, markers of the invention are characterized by molecular weights under the conditions specified herein, particularly as determined by mass spectral analysis

In another aspect, the markers can be characterized by features of the markers' mass spectral signature such as size (including area) and/or shape of the markers' spectral peaks, features including proximity, size and shape of neighboring peaks, etc.

In yet another aspect, the markers can be characterized by affinity binding characteristics, particularly ability to binding to an IMAC Ni adsorbent under specified conditions.

In preferred embodiments, markers of the invention may be characterized by each of such aspects, i.e. molecular weight, mass spectral signature and IMAC Ni absorbent binding.

Protein biomakers of the invention include the following designated herein as Markers I through XIV. Molecular weights as measured by mass spectrometry are also specified for each marker:

| | |
|---|---|
| Marker I (BC1): | having a molecular weight of about 4.3 kD |
| Marker II (BC2): | having a molecular weight of about 8.1 kD |
| Marker III (BC3): | having a molecular weight of about 8.9 kD |
| Marker IV: | having a molecular weight of about 4.5 kD |
| Marker V: | having a molecular weight of about 4.0 kD |
| Marker VI: | having a molecular weight of about 8.3 kD |
| Marker VII: | having a molecular weight of about 17 kD |
| Marker VIII: | having a molecular weight of about 18 kD |
| Marker IX: | having a molecular weight of about 10.2 kD |
| Marker X: | having a molecular weight of about 6.0 kD |
| Marker XI: | having a molecular weight of about 8.4 kD |
| Marker XII: | having a molecular weight of about 7.5 kD |
| Marker XIII: | having a molecular weight of about 9.4 kD |
| Marker XIV: | having a molecular weight of about 16.3 kD |

Markers I through XIV also are characterized by their mass spectral signature. The mass spectra of each of Markers I through XIV are set forth in FIGS. 1A through 1N respectively.

Each of Markers I through XIV also is characterized by its ability to bind to an IMAC Ni adsorbent after washing with phosphate buffered saline, as specified herein.

In one aspect, the present invention provides a method of qualifying breast cancer status in a subject comprising

(a) measuring at least one biomarker in a sample from the subject,

wherein the marker is selected from Marker I (BC1); Marker II (BC2); Marker III (BC3); Marker IV; Marker V; Marker VI; Marker VII; Marker VIII; Marker IX; Marker X; Marker XI; Marker XII; Marker XIII; and Marker XIV, and combinations thereof, and

(b) correlating the measurement with breast cancer status. In certain methods, the measuring step comprises detecting the presence or absence of markers in the sample. In other methods, the measuring step comprises quantifying the amount of marker(s) in the sample. In other methods, the measuring step comprises qualifying the type of biomarker in the sample.

In certain methods, the measuring step comprises detecting the presence or absence of markers in the sample. In other methods, the measuring step comprises quantifying the amount of marker(s) in the sample. In other methods, the measuring step comprises qualifying the type of biomarker in the sample.

The invention also relates to methods wherein the measuring step comprises: depositing a subject sample of blood or a blood derivative on a surface of a substrate comprising capture reagents that bind the protein biomarkers. The subject sample may be optionally fractionated (e.g. on a pH gradient/) prior to such depositing and the collected and selected fractions deposited on the substrate. The blood derivative is, e.g., serum or plasma. In preferred embodiments, the substrate is a SELDI probe comprising an IMAC Ni surface and wherein the protein biomarkers are detected by SELDI. In other embodiments, the substrate is a SELDI probe comprising biospecific affinity reagents that bind such Markers I through XIV as identified above and wherein the protein biomarkers are detected by SELDI. In other embodiments, the substrate is a microtiter plate comprising biospecific affinity reagents that bind one or more of Markers I through XIV as identified above and the one or more protein biomarkers are detected by immunoassay.

In certain embodiments, the methods further comprise managing subject treatment based on the status determined by the method. For example, if the result of the methods of the present invention is inconclusive or there is reason that confirmation of status is necessary, the physician may order more tests. Alternatively, if the status indicates that surgery is appropriate, the physician may schedule the patient for surgery. Likewise, if the result of the test is positive, e.g., the status is late stage breast cancer or if the status is otherwise acute, no further action may be warranted. Furthermore, if the results show that treatment has been successful, no further management may be necessary.

The invention also provides for such methods where the at least one biomarker is measured again after subject management. In these instances, the step of managing subject treatment is then repeated and/or altered depending on the result obtained.

The term “breast cancer status” refers to the status of the disease in the patient. Examples of types of breast cancer statuses include, but are not limited to, the subject's risk of cancer, the presence or absence of disease, the stage of disease in a patient, and the effectiveness of treatment of disease. Other statuses and degrees of each status are known in the art.

Markers of the invention can be resolved from other proteins in a sample by using a variety of fractionation techniques, e.g., chromatographic separation coupled with mass spectrometry, or by traditional immunoassays. In preferred embodiments, the method of resolution involves Surface-Enhanced Laser Desorption/Ionization (“SELDI”) mass spectrometry, in which the surface of the mass spectrometry probe comprises adsorbents that bind the markers.

In other preferred embodiments, comparative protein profiles are generated using the ProteinChip Biomarker System from patients diagnosed with breast cancer and from patients without known neoplastic diseases. A subset of biomarkers was selected based on collaborative results from supervised analytical methods. Preferred analytical methods include ProPeak (3Z Infornatics, SC)., which implements the linear version of the Unified Maximum Separability Analysis (UMSA) algorithm, the Classification And Regression Tree (CART), implemented in Biomarker Pattern Software V4.0 (BPS) (Ciphergen, Calif.).

In a preferred embodiment, the analytical methods are used individually and in cross-comparison to screen for peaks that are most contributory towards the discrimination between breast cancer patients and the non-cancer controls.

In another aspect, the biomarkers were purified and identified. The selected biomarkers, together with the tumor markers CA15.3 and CA27.29, were evaluated individually and in combination through multivariate logistic regression.

While the absolute identity of these markers is not yet known, such knowledge is not necessary to measure them in a patient sample, because they are sufficiently characterized by, e.g., mass and by affinity characteristics. It is noted that molecular weight and binding properties are characteristic properties of these markers and not limitations on means of detection or isolation. Furthermore, using the methods described herein or other methods known in the art, the absolute identity of the markers can be determined.

Preferred methods for detection and diagnosis of cancer comprise detecting at least one or more protein biomarkers in a subject sample, and; correlating the detection of one or more protein biomarkers with a diagnosis of cancer, wherein the correlation takes into account the detection of one or more biomarker in each diagnosis, as compared to normal subjects, wherein the one or more protein markers are selected from Marker I (BC1); Marker II (BC2); Marker III (BC3); Marker IV; Marker V; Marker VII; Marker VIII; Marker IX; Marker X; Marker XI; Marker XII; Marker XIII; and Marker XIV, and combinations thereof.

In a preferred method for detection, diagnosis and determination of the clinical stage of breast cancer, comprises detecting at least one or more protein biomarkers in a subject sample, wherein the protein markers are selected from Marker I (BC1); Marker II (BC2); Marker III (BC3), combinations thereof;

and; correlating the detection of one or more protein biomarkers with a diagnosis of breast cancer, wherein the correlation takes into account the detection of one or more protein biomarkers in each diagnosis, as compared to normal subjects.

In a preferred method for detection, diagnosis and determination of the earliest clinical stages of breast cancer, comprises detecting at least one or more protein biomarkers in a subject sample, wherein the protein markers are selected from Marker I (BC1); Marker II (BC2); Marker III (BC3), and combinations thereof;

and; correlating the detection of one or more protein biomarkers with a diagnosis of breast cancer, wherein the correlation takes into account the detection of one or more protein biomarkers in each diagnosis, as compared to normal subjects. Preferably, the markers are detected at Stage 0, which is the earliest stage of breast cancer. Results showing the sensitivity and specificity of detecting the markers in the early stages are described in the Examples which follow.

In other preferred embodiments, a plurality of the biomarkers are detected, preferably at least one of the biomarkers is detected, more preferably at least two of the biomarkers are detected, most preferably at least three of the biomarkers are detected. The most preferred markers are:

| | |
|---|---|
| Marker I (BC1): | having a molecular weight of about 4.3 kD |
| Marker II (BC2): | having a molecular weight of about 8.1 kD |
| Marker III (BC3): | having a molecular weight of about 8.9 kD |

In a preferred method for diagnosing and differentiating between the different malignant stages of cancer, the method comprises using a biochip array to generate a first set of data representative of the first set of biological markers; and evaluating the first set of data detecting at least one or more protein biomarkers in a subject sample, and; correlating the detection of one or more protein biomarkers with a progressive malignant stage of cancer as compared to normal subjects.

In one aspect of the invention the method comprises detecting one or more protein biomarkers are used in diagnosing and differentiating between the different malignant stages of cancer; wherein, the one or more protein markers are selected from Marker I (BC1); Marker II (BC2); Marker III (BC3); Marker IV; Marker V; Marker VII; Marker VIII; Marker IX; Marker X; Marker XI; Marker XII; Marker XIII; and Marker XIV, and combinations thereof.

In another aspect, the present invention provides for a method for diagnosing and differentiating between the different malignant stages of breast cancer, wherein the method comprises:
detecting at least one or more protein biomarkers in a subject sample, and; correlating the detection of one or more protein biomarkers with a diagnosis of breast cancer, wherein the correlation takes into account the detection of one or more protein biomarkers in each diagnosis, as compared to normal subjects.

In another aspect of the invention, a single biomarker is used to differentiate between the different malignant stages of cancer. Also provided is a single biomarker to differentiate between the different malignant stages of cancer in combination with one or more known cancer biomarkers for diagnosing cancer such as, for example, the breast cancer markers CA 15.3 and CA 27.29. It is preferred that one or more protein biomarkers are used in comparing protein profiles from patients susceptible to, or suffering from cancer, such as breast cancer, with normal subjects.

In another aspect of the invention, the patient sample is selected from the group consisting of blood, blood plasma, serum, urine, tissue, cells, organs and vaginal fluids.

Preferred detection methods include use of a biochip array. Biochip arrays useful in the invention include protein and nucleic acid arrays. One or more markers are immobilized on the biochip array and subjected to laser ionization to detect the molecular weight of the markers. Analysis of the markers is, for example, by molecular weight of the one or more markers against a threshold intensity that is normalized against total ion current. Preferably, logarithmic transformation is used for reducing peak intensity ranges to limit the number of markers detected.

In another preferred method, data is generated on immobilized subject samples on a biochip array, by subjecting said biochip array to laser ionization and detecting intensity of signal for mass/charge ratio; and, transforming the data into computer readable form; and executing an algorithm that classifies the data according to user input parameters, for detecting signals that represent markers present in breast cancer patients and are lacking in non-cancer subject controls.

Preferably the biochip surfaces are, for example, ionic, anionic, comprised of immobilized nickel ions comprised of a mixture of positive and negative ions, comprises one or more antibodies, single or double stranded nucleic acids, comprises proteins, peptides or fragments thereof, amino acid probes, comprises phage display libraries.

In other preferred methods one or more of the markers are detected using laser desorption/ionization mass spectrometry, comprising, providing a probe adapted for use with a mass spectrometer comprising an adsorbent attached thereto, and; contacting the subject sample with the adsorbent, and; desorbing and ionizing the marker or markers from the probe and detecting the deionized/ionized markers with the mass spectrometer.

Preferably, the laser desorption/ionization mass spectrometry comprises, providing a substrate comprising an adsorbent attached thereto; contacting the subject sample with the adsorbent; placing the substrate on a probe adapted for use with a mass spectrometer comprising an adsorbent attached thereto; and, desorbing and ionizing the marker or markers from the probe and detecting the desorbed/ionized marker or markers with the mass spectrometer.

In another embodiment, various compositions are provided to further aid in the diagnosis of breast cancer:

A composition comprising Marker I and one more biomarkers selected from Markers II through XIV.

A composition comprising Marker II and one more biomarkers selected from Markers I, III, through XIV.

A composition comprising Marker III and at least one more biomarkers selected from Markers I, II, IV through XIV.

A composition comprising Marker IV and at least one more biomarkers selected from Markers I, II, III, V through XIV.

A composition comprising Marker V and at least one more biomarkers selected from Markers I, II, III, IV, VI through XIV.

A composition comprising Marker VI and one more biomarkers selected from Markers I, II, III, IV, V through XIV.

A composition comprising Marker VII and one more biomarkers selected from Markers I, II, III, IV, V, VI, VIII through XIV.

A composition comprising Marker VIII and one more biomarkers selected from Markers I, II, III, IV, V, VI, VII through XIV.

A composition comprising Marker IX and one more biomarkers selected from Markers I, II, III, IV, V, VI, VII, VII, X through XIV.

A composition comprising Marker X and one more biomarkers selected from Markers I through IX, XI through XIV.

A composition comprising Marker XI and one more biomarkers selected from Markers I through X, XII through XIV.

A composition comprising Marker XII and one more biomarkers selected from Markers I through XI, XIII through XIV.

A composition comprising Marker XIII and one more biomarkers selected from Markers I through XII, XIV.

A composition comprising Marker XIV and one more biomarkers selected from Markers I through XIII. Preferably, in these compositions, the markers are substantially pure and/or isolated e.g. from a serum sample.

For the mass values of the markers disclosed herein, the mass accuracy of the spectral instrument is considered to be about within +/−0.15 percent of the disclosed molecular weight value. Additionally, to such recognized accuracy variations of the instrument, the spectral mass determination can vary within resolution limits of from about 400 to 1000 m/dm, where m is mass and dm is the mass spectral peak width at 0.5 peak height. Those mass accuracy and resolution variances associated with the mass spectral instrument and operation thereof are reflected in the use of the term “about” in the disclosure of the mass of each of Markers I through XIV. It is also intended that such mass accuracy and resolution variances and thus meaning of the term “about” with respect to the mass of each of markers I through XIV is inclusive of variants of the markers as may exist due to sex and/or ethnicity of the subject and the particular cancer or origin or stage thereof.

In the discovery of the biomarkers for breast cancer using the methods of this invention, the preferred analysis of the date is by logarithmic transformation for reducing peak intensity ranges to limit the number of markers detected and data obtained from the peak intensities of each sample are projected as individual points onto a three-dimensional component space. The component spaces are linear combinations of the peak intensities. Each component space corresponds to directions along which about two pre-specified groups of data achieve maximum separation as determined by an interactive three dimensional display on a computer monitor. A significance score for each individual mass peak is computed and each individual mass peak is ranked according to their collective contribution towards the maximal separation of two pre-specified groups of data. A significance score may be positive or negative values, wherein a positive score correlates with an increased expression of the corresponding mass peak obtained from a patient sample with cancer and a negative score correlates with a decreased expression of a corresponding mass peak obtained from a cancer patient sample.

The immobilized molecules are subjected to laser ionization to detect mass peaks of each biomarker and the mass peaks of the one or more markers are analyzed against a threshold intensity that is normalized against total ion current. Preferred selected mass peaks are between about 2 K to about 150 K.

In a preferred embodiment, a fixed percentage of biomarker samples are randomly excluded during the analysis of mass peaks, wherein a median and mean rank is determined for each peak. The analysis is run at least about 100 times.

In another preferred embodiment, a method for predicting mass peaks that are representative of a biomarker for detecting and differentiating between the progressive stages of cancer, is provided for, said method comprising:

obtaining samples from normal subjects and subjects suffering from cancer, and;

providing a biochip array for evaluating the mass peaks of said samples, wherein;

said biochip array comprising a chemically modified metal affinity surface having stably attached thereto a plurality of molecules capable of selective binding to at least one member of the group consisting of proteins, peptides or fragments thereof, and;

using the samples from a normal subject and cancer patient subjects to obtain a first set of data representative of the first set of biomarkers, and;

evaluating the first set of data detecting at least one or more protein biomarkers in a subject sample, and;

correlating the detection of one or more protein biomarkers with a progressive malignant stage of cancer, such as breast cancer as compared to normal subjects.

In one aspect a set of control data is generated, comprising:
generating a control set of biomarkers representative of a normal subject;
using the biochip array to generate a control set of data representative of the control set of biomarkers; and,
comparing the first and control sets of data to predict which mass peaks are representative of a biomarker that differentiates between the different malignant stages of cancer progression.
In another aspect the method further comprising:
generating a second set of biomarkers representative of a patient suffering from cancer, and;
using the biochip array to generate a second set of data representative of a first stage of cancer; and,
comparing the second and control sets of data to predict which mass peaks are representative of a biomarker that differentiates between a first and second stage of cancer progression.

The method is repeated at least one or more times until a set of data is obtained which is used to predict the mass peaks of any potential biomarker representative of a certain stage of a malignant cancer.

In another preferred embodiment a biomarker database is constructed which contains a plurality of data sets representative of the different stages of breast cancer; and by comparing the test set of data with the database to predict the mass peaks which are potential biomarkers for detecting at least one stage of breast cancer.

The database is used to predict mass peaks which are potential biomarkers for detecting any stage of breast cancer and for mining of data from the database for evaluation and prediction of potential biomarkers which differentiate between different stages of different cancers.

In another preferred embodiment, tissue samples from cancer patients are analyzed and compared to normal subjects by obtaining data sets of mass peaks which are used to determine potential biomarkers which are present in pre-invasive tumors such as a breast tumor.

Other aspects of the invention are described infra.

### BRIEF DESCRIPTION OF THE FIGURES

FIGS. 1A through 1N show mass spectra of Markers I through XIV respectively. In those Figures, the mass spectral peak of the specified marker is designated within the depicted spectra with an arrow. The Figure designation is set above each of the referred to spectra.

FIG. 2 shows a representative mass peak spectrum obtained by SELDI analysis of serum proteins retained on an IMAC-Ni²+ chip. The upper panel shows the spectrum view; the lower panel shows the pseudo-gel view of the same spectrum of M/Z (mass-dependent velocities) between 4,000 and 10,000.

FIG. 3 shows the results of logarithmic transformation on data variance reduction and equalization.

FIGS. 4A-4B show a 3 dimensional-UMSA-component plot of stages 0-I breast cancer (darker squares) versus non-cancer (white squares).

FIG. 4A shows illustrative results of separation achieved using UMSA derived liner combination of all 147 peaks.

FIG. 4B shows illustrative results of separation achieved using UMSA derived liner combination using the three selected peaks.

FIG. 5 is a graph showing fifteen peaks with top mean ranks and minimal rank standard deviations derived from ProPeak Bootstrap Analysis. Horizontal line at 7.0 was the minimum rank standard deviation computed by applying the same procedure to a randomly generated data set that simulated the distribution of the original data.

FIGS. 6A-6B are graphs showing a plot of absolute values of the relative significance scores of selected peaks based on contribution towards the separation between stages 0-I breast cancer and the non-cancer controls.

FIG. 6A shows the results of 15 peaks selected from ProPeak Bootstrap Analysis with rank standard deviation <7.0.

FIG. 6B is a graph showing re-evaluated scores of the selected top 4 peaks from FIG. 5A.

FIG. 7 is a graph showing receiver-operating-characteristic (ROC) curve analysis of BC1, BC2, BC3, and logistic regression derived composite index. p-values from AUC (Area-under-curve) comparison between each individual biomarkers and the Composite Index are listed in the figure.

FIG. 8A-8B are scatter plots showing the distribution of the selected biomarker(s) across all diagnostic groups including clinical stages of the cancer patients.

FIG. 8A is a scatter plot showing the results obtained with BC3 alone.

FIG. 8B is a scatter plot showing the results of a logistic regression derived composite index using BC1, BC2 and BC3.

FIG. 9 shows a panel of three 2 dimensional scatter plots depicting distributions of all patient samples.

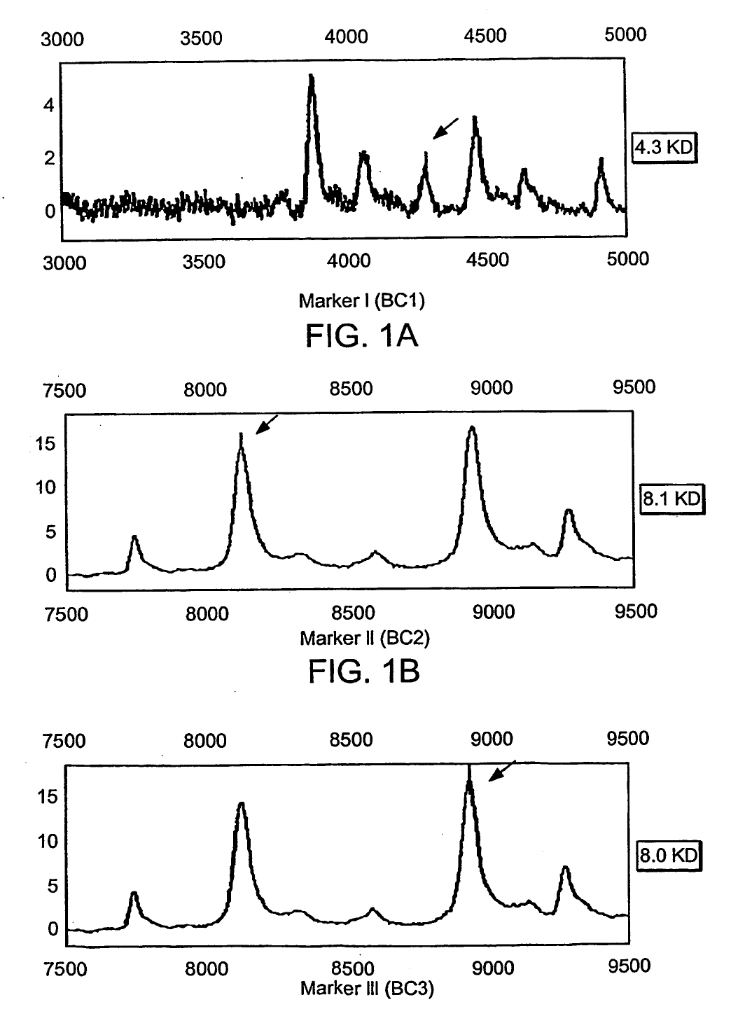

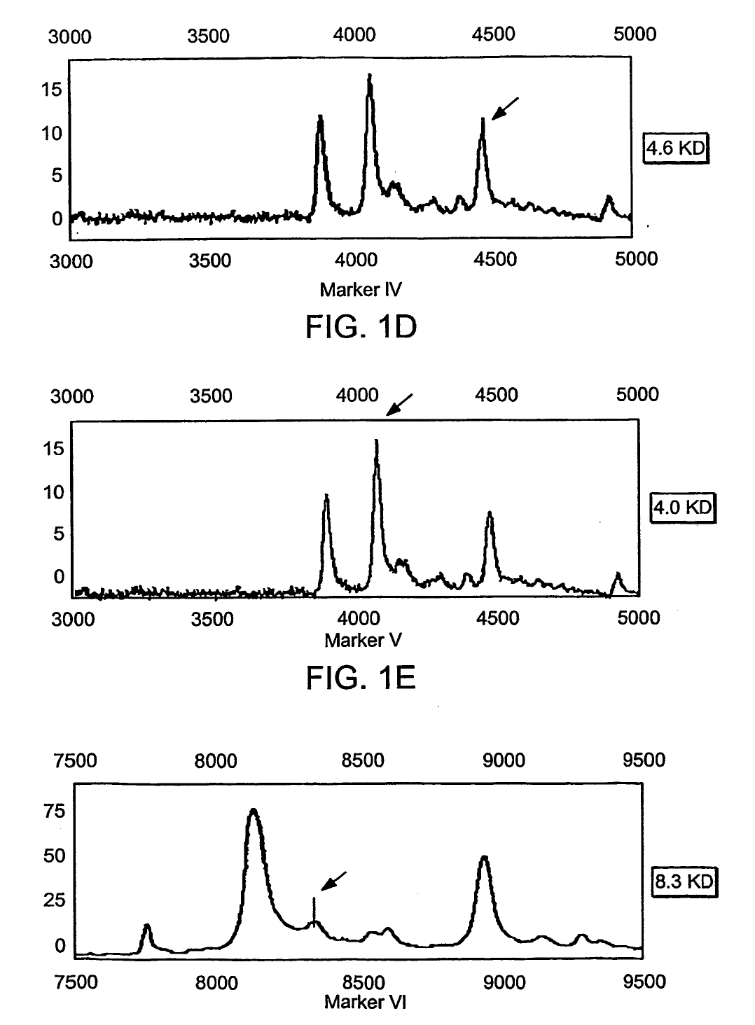

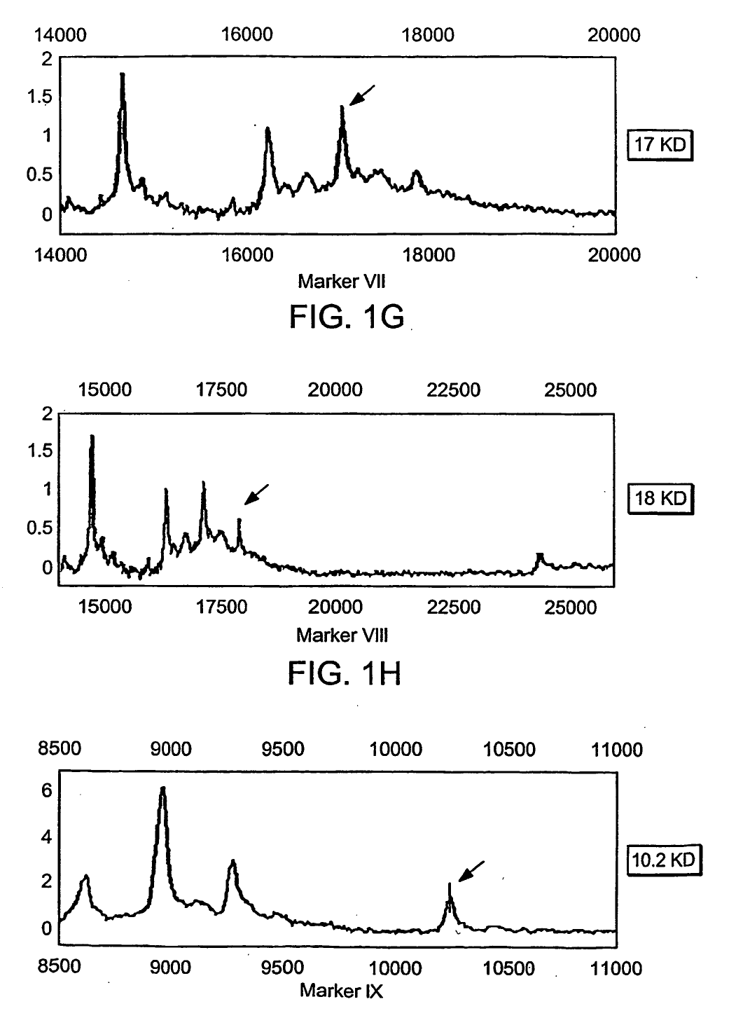

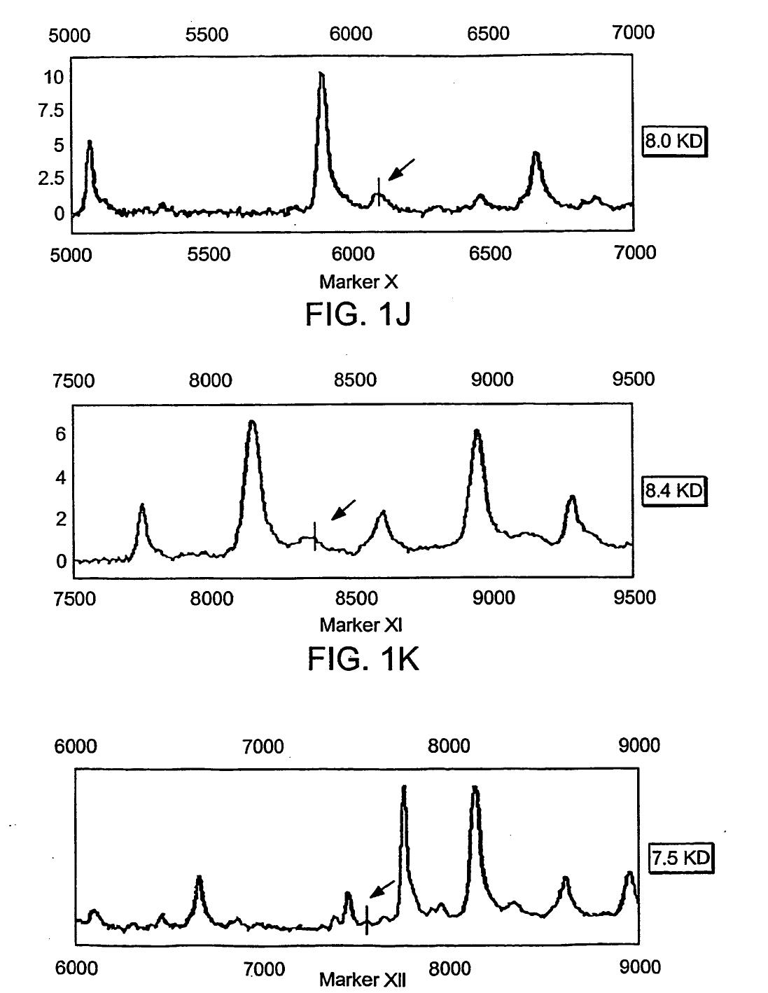

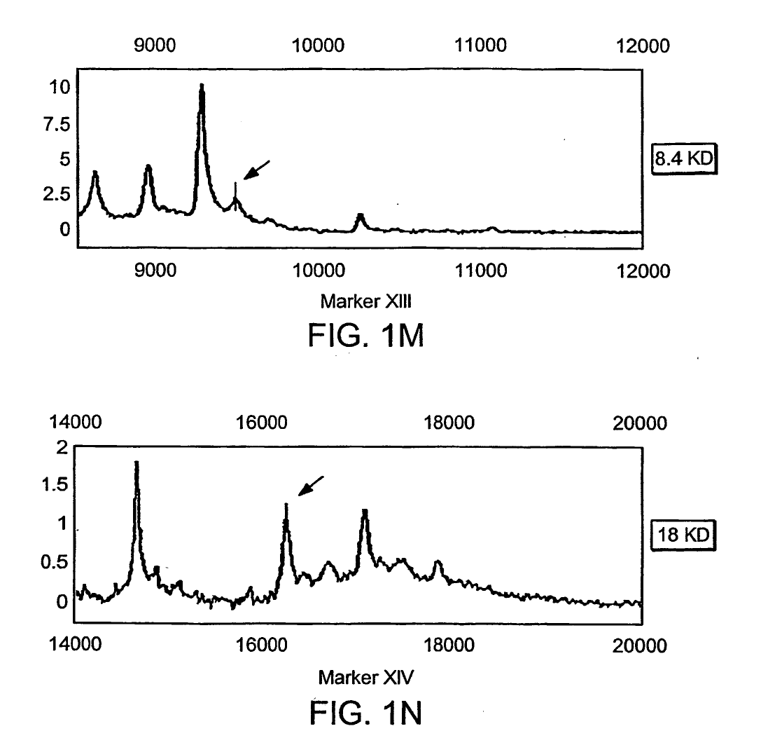

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd—Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ ionization source and thereafter contributing to desorption and ionization of analyte molecules in contact therewith. The phrase includes molecules used in MALDI , frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface and, therefore, function as probes.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 biochip has quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu⁺⁺ and Ni++, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application US20030032043A1 (Pohl and Papanu, “Latex Based Adsorbent Chip,” Jul. 16, 2002) and U.S. patent application 60/350,110 (Um et al., “Hydrophobic Surface Chip,” Nov. 8, 2001).

Upon capture on a reaction medium or substrate such as a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

“Marker” in the context of the present invention refers to a polypeptide (of a particular apparent molecular weight) which is differentially present in a sample taken from patients having human cancer as compared to a comparable sample taken from control subjects (e.g., a person with a negative diagnosis or undetectable cancer, normal or healthy subject).

The phrase “differentially present” refers to differences in the quantity and/or the frequency of a marker present in a sample taken from patients having human cancer as compared to a control subject. For examples, a marker can be a polypeptide which is present at an elevated level or at a decreased level in samples of human cancer patients compared to samples of control subjects. Alternatively, a marker can be a polypeptide which is detected at a higher frequency or at a lower frequency in samples of human cancer patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in the human cancer patients' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of human cancer. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without human cancer. A control amount can be either in absolute amount (e.g., μg/ml) or a relative amount (e.g., relative intensity of signals).

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker Br 1 from specific species such as rat, mouse, or human can be selected to obtain only those polyclonal antibodies that are specifically immunoreactive with marker Br 1 and not with other proteins, except for polymorphic variants and alleles of marker Br 1. This selection may be achieved by subtracting out antibodies that cross-react with marker Br 1 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

### DETAILED DESCRIPTION OF THE INVENTION

The present invention relates to a method for identification of tumor biomarkers for breast cancer, with high specificity and sensitivity.

Fifteen (14) biomarkers were identified that are associated with breast cancer disease status. The corresponding proteins or fragments of proteins for the fourteen biomarkers are represented as intensity peaks in SELDI (surface enhanced laser desorption/ionization) protein chip/mass spectra with molecular masses centered around the following values:

| | |
|---|---|
| Marker I (BC1): | 4283 daltons |
| Marker II (BC2): | 8126 daltons |
| Marker III (BC3): | 8932 daltons |
| Marker IV: | 4465 daltons |
| Marker V: | 4060 daltons |
| Marker VI: | 8322 daltons |
| Marker VII: | 17046 daltons |
| Marker VIII: | 17696 daltons |
| Marker IX: | 10240 daltons |
| Marker X: | 5891 daltons |
| Marker XI: | 8426 daltons |
| Marker XII: | 7541 daltons |
| Marker XIII: | 9413 daltons |
| Marker XIV: | 16244 daltons. |

These masses for Markers I through XIV are considered accurate to within 0.15 percent of the specified value as determined by the disclosed SELDI-mass spectroscopy protocol.

We have found that Markers I through XIV are differentially present in quantity and/or frequency in sample taken from patients having human breast cancer as compared to a control subject as follows, particularly the marker being up regulated in cancer patient sample (i.e. elevated level in samples of breast cancer patients compared to samples from control subjects) or the marker being down regulated (i.e. decreased level in samples of breast cancer patients compared to samples from control subjects):

| | |
|---|---|
| Marker I (BC1): | down regulated in cancer patient sample |
| Marker II (BC2): | up regulated in cancer patient sample |
| Marker III (BC3): | up regulated in cancer patient sample |
| Marker IV: | up regulated in cancer patient sample |
| Marker V: | up regulated in cancer patient sample |
| Marker VI: | up regulated in cancer patient sample |
| Marker VII: | up regulated in cancer patient sample |
| Marker VIII: | up regulated in cancer patient sample |
| Marker IX: | down regulated in cancer patient sample |
| Marker X: | down regulated in cancer patient sample |
| Marker XI: | up regulated in cancer patient sample |
| Marker XII: | down regulated in cancer patient sample |
| Marker XIII: | up regulated in cancer patient sample |
| Marker XIV: | up regulated in cancer patient sample |

The association between Markers I through XIV and the absence or presence of breast cancer was established in patients with breast cancer before tumor resection and treatment at various stages (n=103), women with benign breast diseases (n=25) and healthy women (n=41) without known neoplastic diseases. The high specificity and sensitivity of the method used for identifying the biomarkers that differentiate between the different stages of breast cancer is underscored by using only three of these biomarkers, 4283 (BC1), 8126 (BC2) and 8932 (BC3), to correctly identify 93% of breast cancer patients at different stages: Stage 0/I (93%), stage II (85%) and stage III (94%). Using only one biomarker (BC3), correct identification 85% of breast cancer patients with stage 0/I (88%), stage II (78%) and stage III (92%) was achieved.

In particular, simultaneous analysis of protein profiles of 169 serum samples of subjects with or without breast cancer using was carried out and the results demonstrate the high specificity and selectivity of the methods described herein. Out of the 169 serum samples of subjects, three discriminating biomarkers were identified, the combination of which achieved both high sensitivity (93%) and high specificity (91%) in detecting breast cancer from the non-cancer controls.

As discussed above, Markers I through XIV also may be characterized based on affinity for an adsorbent, particularly binding to an immobilized Ni chelate (IMAC) substrate surface under the conditions specified under ProteinChip Analysis of the General Comments of the Examples which follow, which conditions include 30 μl of 8M urea, 1% CHAPS in PBS, PH 7.4 is added to a 20 μl serum sample; the diluted sample is vortexed at 4° C. for 15 minutes and diluted 1:40 in PBS; immobilized metal affinity capture chips (IMAC3) are activated with 50 mM NiSO₄ according to manufacturer's instructions (Ciphergen Biosystems, Inc., CA); 50 μl of the diluted serum samples are applied to each spot on the ProteinChip array by using a 96 well bioprocessor (Ciphergen Biosystems, Inc., CA); after binding at room temperature for 60 minutes on a platform shaker, the array is washed twice with 100 μl of PBS for 5 minutes followed by two rinses with 100 μl of dH₂O; binding can be detected with a mass reader. References herein that a particular protein marker can be characterized as binding to an IMAC Ni adsorbent indicates detection of binding of the marker with a serum sample processed under those conditions.

The following illustrative example of the invention for identification of biomarkers is not meant to limit or construe the invention in any way. To identify serum biomarkers with potential for early detection of breast cancer, protein profiles of specimens from cancer patients at stages 0 and I were compared to those from the non-cancer controls. The selected biomarkers were then tested using data from breast cancer patients at Stage II and III, which were not included in the biomarker selection process. High-throughput profiling of complex protein expression patterns greatly facilitates the screening of a large number of potential markers simultaneously. The UMSA algorithm provided an efficient model to rank a large number of peaks collectively according to their contribution to the separation of two predefined diagnostic groups. The ProPeak Bootstrap module introduced random perturbations in multiple runs to test the consistency of the top-ranked peaks, measured by the standard deviation of computed ranks from multiple runs. In order to establish an upper bound cutoff value on a peak's rank standard deviation for its performance not to be considered as purely by chance, the same bootstrap procedure was applied to a randomly generated data set that simulates the distribution of the real data. The minimum value of rank standard deviations from such “simulated peaks” indicates the level of consistency that a peak might achieve by random chance. This minimum value was used as the cutoff to help to reduce the original 147 peaks to a subset of 15 top-ranked peaks for further consideration. The performance of such peaks should be less likely due to random artifacts in the data.

For simplicity, the composite index was derived by simple multivariate logistic regression. When these selected biomarkers are further validated, more complex and nonlinear classification models may be employed to combine the multiple biomarkers. The use of complex modeling methods on carefully screened and tested biomarkers should in general offer a more robust performance than the direct application of such methods on raw data from a large number of mass peaks. The discriminatory power of the selected biomarkers was verified using stages II-III data as an independent test set. The bootstrap cross-validation estimation of performance offers statistical confidence on the generalizability of these biomarkers over future data Of the three biomarkers selected, no significant correlation was found between the level of these markers and the tumor size or lymph node metastasis. The discriminatory power of these markers is therefore most likely reflecting the malignant nature of the tumor rather than the progression of it.

As used herein, “tumor stage” or “tumor progression” refers to the different clinical stages of the tumor. Clinical stages of a tumor are defined by various parameters which are well-established in the field of medicine. Some of the parameters include morphology, size of tumor, the degree in which it has metastasized through the patient's body and the like.

Cancer in humans appears to be a multi-step process which involves progression from pre-malignant to malignant to metastatic disease which ultimately kills the patient. Epidemiological studies in humans have established that certain pathologic conditions are “pre-malignant” because they are associated with increased risk of malignancy. There is precedent for detecting and eliminating pre-invasive lesions as a cancer prevention strategy: dysplasia and carcinoma in-situ of the uterine cervix are examples of pre-malignancies which have been successfully employed in the prevention of cervical cancer by cytologic screening methods. Unfortunately, because the breast cannot be sampled as readily as cervix, the development of screening methods for breast pre-malignancy involves more complex approaches than cytomorphologic screening now currently employed to detect cervical cancer.

Pre-malignant breast disease is also characterized by an apparent morphological progression from atypical hyperplasias, to carcinoma in-situ (pre-invasive cancer) to invasive cancer which ultimately spreads and metastasizes resulting in the death of the patient. Careful histologic examination of breast biopsies has demonstrated intermediate stages which have acquired some of these characteristics but not others. Detailed epidemiological studies have established that different morphologic lesions progress at different rates, varying from atypical hyperplasia (with a low risk) to comedo ductal carcinoma-in-situ (DCIS) which progresses to invasive cancer in a high percentage of patients (London et al, 1991; Page et al, 1982; Page et al, 1985; Page et al, 1991; and Page et al, 1978). Family history is also an important risk factor in the development of breast cancer and increases the relative risk of these pre-malignant lesions (Dupont et al, 1985; Dupont et al, 1993; and, London et al, 1991). Of particular interest is non-comedo carcinoma-in-situ which is associated with a greater than ten-fold increased relative risk of breast cancer compared to control groups (Ottesen et al, 1992; Page et al, 1982). Two other reasons besides an increased relative risk support the concept that DCIS is pre-malignant: 1) When breast cancer occurs in these patients it regularly occurs in the same region of the same breast where the DCIS was found; and 2) DCIS is frequently present in tissue adjacent to invasive breast cancer (Ottesen et al, 1992; Schwartz et al, 1992). For these reasons DCIS very likely represents a rate-limiting step in the development of invasive breast cancer in women.

I. Examples of Preferred Embodiments

In a preferred embodiment, the invention provides methods for aiding a human cancer diagnosis using one or more markers, for example Markers 4283 (BC1), 8126 (BC2) and 8932 (BC3). These markers can be used alone, in combination with other markers in any set, or with entirely different markers (e.g., CA15.3 and CA27.29) in aiding human cancer diagnosis. The markers are differentially present in samples of a human cancer patient, for example breast cancer patient, and a normal subject in whom human cancer is undetectable. For example, some of the markers are expressed at an elevated level and/or are present at a higher frequency in human cancer patients than in normal subjects. Therefore, detection of one or more of these markers in a person would provide useful information regarding the probability that the person may have human cancer and also be able to determine the clinical stage of the tumor.

Accordingly, embodiments of the invention include methods for diagnosing and differentiating between the different malignant stages of cancer by (a) using a biochip array to generate a first set of data representative of the first set of biological markers; (b) evaluating the first set of data detecting at least one or more protein biomarkers in a subject sample; (c) correlating the detection of one or more protein biomarkers with a progressive malignant stage of cancer as compared to normal subjects. The correlation may take into account the amount of the marker or markers in the sample compared to a control amount of the marker or markers (up or down regulation of the marker or markers) (e.g., in normal subjects in whom human cancer is undetectable). The correlation may take into account the presence or absence of the markers in a test sample and the frequency of detection of the same markers in a control. The correlation may take into account both of such factors to facilitate determination of whether a subject has a human cancer or not.

Any suitable samples can be obtained from a subject to detect markers. Preferably, a sample is a blood serum sample from the subject. If desired, the sample can be prepared as described above to enhance detectability of the markers. For example, to increase the detectability of markers 4283 (BC1), 8126 (BC2) and 8932 (BC3), a blood serum sample from the subject can be preferably fractionated by, e.g., Cibacron blue agarose chromatography and single stranded DNA affinity chromatography, anion exchange chromatography and the like. Sample preparations, such as pre-fractionation protocols, is optional and may not be necessary to enhance detectability of markers depending on the methods of detection used. For example, sample preparation may be unnecessary if antibodies that specifically bind markers are used to detect the presence of markers in a sample.

Any suitable method can be used to detect a marker or markers in a sample. For example, gas phase ion spectrometry or an immunoassay can be used as described above. Using these methods, one or more markers can be detected. Preferably, a sample is tested for the presence of a plurality of markers. Detecting the presence of a plurality of markers, rather than a single marker alone, would provide more information for the diagnostician. Specifically, the detection of a plurality of markers in a sample would increase the percentage of true positive and true negative diagnoses and would decrease the percentage of false positive or false negative diagnoses.

The detection of the marker or markers is then correlated with a probable diagnosis of human cancer. In some embodiments, the detection of the mere presence or absence of a marker, without quantifying the amount of marker, is useful and can be correlated with a probable diagnosis of human cancer and the determination of the clinical stage of the tumor. For example, use of only three of these biomarkers, 4283 (BC1), 8126 (BC2) and 8932 (BC3), 93% of breast cancer patients were correctly identified at the following stages: Stage 0/I (93%), stage II (85%) and stage m (94%). With only one biomarker (BC3), 85% of breast cancer patients with stage 0/I (88%), stage II (78%) and stage III (92%), were correctly identified. Thus, a mere detection of one or more of these markers in a subject being tested indicates that the subject has progressed to a different clinical stage of the tumor.

In other embodiments, the detection of markers can involve quantifying the markers to correlate the detection of markers with a probable diagnosis of human cancer. Thus, if the amount of the markers detected in a subject being tested is higher compared to a control amount, then the subject being tested has a higher probability of having a human cancer.

Similarly, in another embodiment, the detection of markers can further involve quantifying the markers to correlate the detection of markers with a probable diagnosis of human cancer wherein the markers are present in lower quantities in blood serum samples from human cancer patients than in blood serum samples of normal subjects. Thus, if the amount of the markers detected in a subject being tested is lower compared to a control amount, then the subject being tested has a higher probability of having a human cancer.

When the markers are quantified, it can be compared to a control. A control can be, e.g., the average or median amount of marker present in comparable samples of normal subjects in whom human cancer is undetectable. The control amount is measured under the same or substantially similar experimental conditions as in measuring the test amount. For example, if a test sample is obtained from a subject's blood serum sample and a marker is detected using a particular probe, then a control amount of the marker is preferably determined from a serum sample of a patient using the same probe. It is preferred that the control amount of marker is determined based upon a significant number of samples from normal subjects who do not-have human cancer so that it reflects variations of the marker amounts in that population.

Data generated by mass spectrometry can then be analyzed by a computer software. The software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human cancer and determines the closeness of fit between the two signals. The software also can include code indicating which the test sample is closest to, thereby providing a probable diagnosis.

In accordance with the present invention, the methods described herein, pre-invasive or even benign tumors may be diagnosed by identifying the biomarkers which cause a pre-invasive tumor to progress to a malignant tumor. The type of biomarkers identified and amounts of biomarker may correlate with the jump from a pre-invasive tumor to a malignant stage tumor. Therapy such as immediate excision of the tumor or therapies such as chemotherapy or radiation therapy can be implemented prior to the tumor becoming invasive. The identification of the pre-invasive biomarkers can be used in diagnosis with conventional methods such as, for example, in breast cancer, use of mammograms.

The present invention thus provides for the immediate identification of a pre-invasive tumor by identifying the biomarkers associated with such tumors and the patient may be given life-saving therapy. Furthermore, the costs of long term treatment of cancer patients will also be reduced.

More specifically, the present invention is based upon, the discovery of protein markers that are differentially present in samples of human cancer patients and control subjects, and the application of this discovery in methods for aiding a human cancer diagnosis and tumor stage progression. Some of these protein markers are found at an elevated level and/or more frequently in samples from human cancer patients compared to a control (e.g., women in whom human cancer is undetectable). Accordingly, the amount of one or more markers found in a test sample compared to a control, or the mere detection of one or more markers in the test sample provides useful information regarding probability of whether a subject being tested has human cancer or not.

The protein markers of the present invention have a number of other uses. For example, the markers can be used to screen for compounds that modulate the expression of the markers in vitro or in vivo, which compounds in turn may be useful in treating or preventing human cancer in patients. In another example, markers can be used to monitor responses to certain treatments of human cancer. In yet another example, the markers can be used in the heredity studies. For instance, certain markers may be genetically linked. This can be determined by, e.g., analyzing samples from a population of human cancer patients whose families have a history of human cancer. The results can then be compared with data obtained from, e.g., human cancer patients whose families do not have a history of human cancer. The markers that are genetically linked may be used as a tool to determine if a subject whose family has a history of human cancer is pre-disposed to having human cancer.

In another aspect, the invention provides methods for detecting markers which are differentially present in the samples of a human cancer patient and a control (e.g., women in whom human cancer is undetectable). The markers can be detected in a number of biological samples. The sample is preferably a biological fluid sample. Examples of a biological fluid sample useful in this invention include blood, blood serum, plasma, nipple aspirate, urine, tears, saliva, etc. Because all of the markers are found in blood serum, blood serum is a preferred sample source for embodiments of the invention.

Any suitable methods can be used to detect one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

In preferred embodiments, the method of resolution of involves Surface-Enhanced Laser Desorption/Ionization (“SELDI”) mass spectrometry, in which the surface of the mass spectrometry probe comprises adsorbents that bind the markers. SELDI is an affinity based MS method in which proteins are selectively adsorbed to a chemically modified surface (ProteinChip® arrays, Ciphergen Biosystems, Inc., Fremont Calif.), and impurities are removed by washing with buffer. By combining an. array of different surfaces and wash conditions, high speed, high-resolution chromatographic separations are achieved on chip. M. Merchant et al., Electrophoresis, 2000; 21:1164-67.

SELDI TOF-MS offers high-throughput protein profiling. Like many other types of high-throughput expression data, protein array data are often characterized by a large number of variables (the mass peaks) relative to a small sample size (the number of specimens). An important issue in analyzing such data to screen for disease-associated biomarkers is to extract as much information as possible from a limited number of samples and to avoid selecting biomarkers whose performances are influenced mostly by non-disease related artifacts in the data. The effective and appropriate use of bioinformatics tools becomes very critical.

In other preferred embodiments, immobilized metal affinity ProteinChip arrays and SELDI to screen for potential serum biomarkers for early detection of breast cancer are used for high through put screening. For example, a total of 169 retrospective serum samples from patients with or without breast cancer were obtained from Johns Hopkins Clinical Chemistry Serum Banks and analyzed simultaneously. Proteins bound to the chelated metal (through histidine, tryptophan, cysteine or phosphorylated amino acids) were analyzed on a PBS-II mass reader (Ciphergen Biosystems, Inc., Fremont, Calif.). The complex protein profiles were analyzed using a collection of bioinformatics tools. A panel of three biomarkers was selected based on their consistently significant contribution to the optimal separation of stages 0-I breast cancer patients versus the non-cancer controls (Healthy+Benign). The effectiveness of the selected biomarkers was then tested using independent data from stages II-III breast cancer patients and through bootstrap cross-validation.

II. Preparation of Markers

Preferably, the sample is prepared prior to detection of biomarkers. Typically, this involves collection of a sample from a subject to be tested. The sample can be any biological sample from the subject. Preferably is a biological fluid or a derivative thereof such as blood, plasma serum, urine, lymphatic fluid or fluid from ductal lavage. Most preferably, the sample is serum.

It may be useful to pre-fractionate the sample and to collect fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis. However, the markers of the present invention are detectable by SELDI after no more fractionation than isolating serum from blood.

In one embodiment, a sample can be pre-fractionated according to size of proteins in a sample using size exclusion chromatography. For a biological sample wherein the amount of sample available is small, preferably a size selection spin column is used. For example, a K30 spin column (available from Princeton Separation, Ciphergen Biosystems, Inc., etc.) can be used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by gas phase ion spectrometry for the detection of markers.

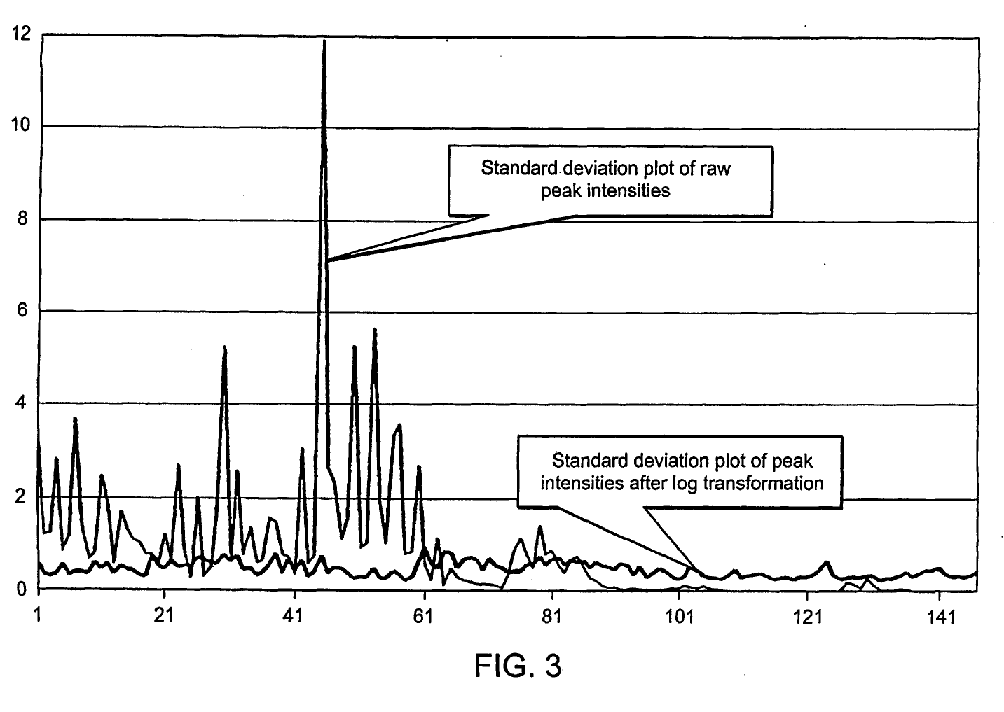

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's (see FIG. 3 and Example section VI B). Anion exchange chromatography allows separation of biomolecules in a sample that are more negatively charged from other types of biomolecules. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

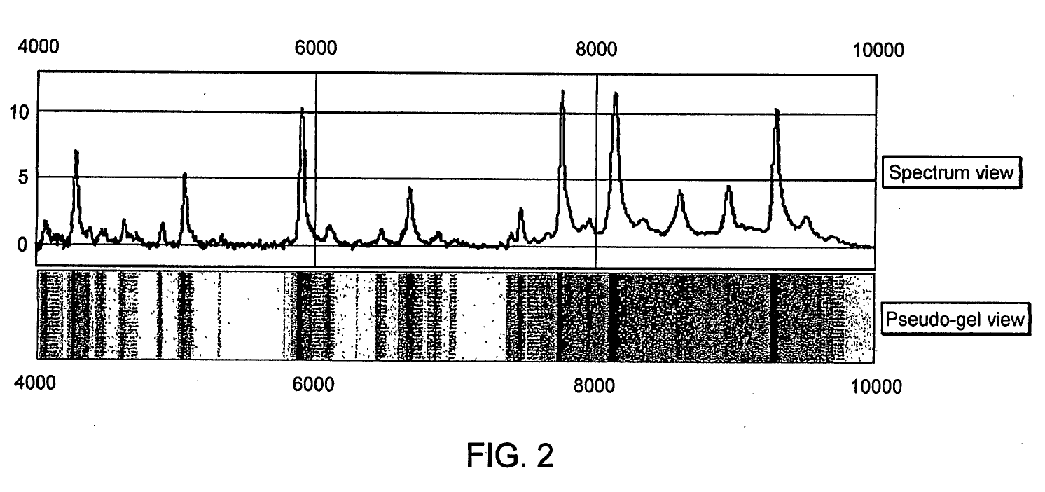

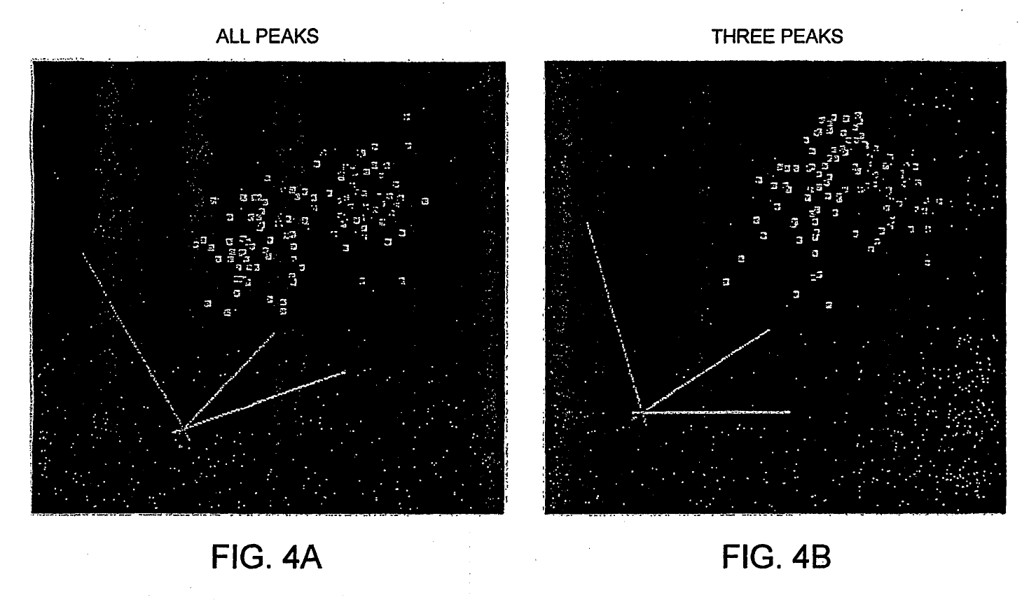

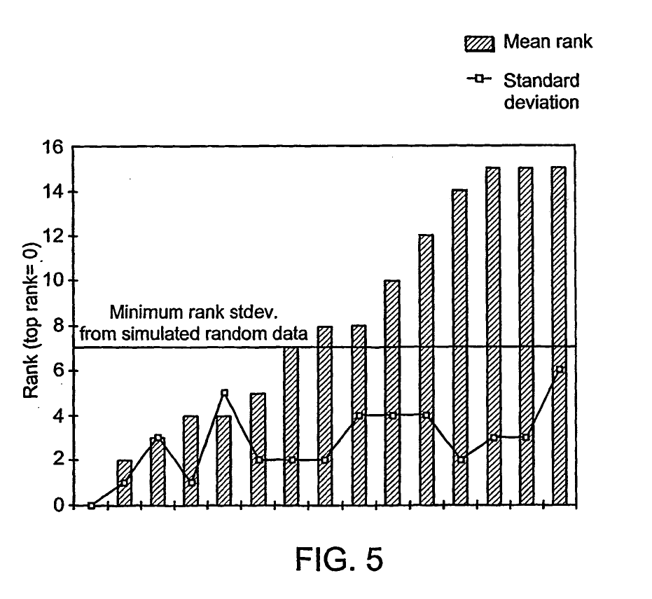

In yet another embodiment, a sample can be pre-fractionated by removing proteins that are present in a high quantity or that may interfere with the detection of markers in a sample. For example, in a blood serum sample, serum albumin is present in a high quantity and may obscure the analysis of markers. Thus, a blood serum sample can be pre-fractionated by removing serum albumin. Serum albumin can be removed using a substrate that comprises adsorbents that specifically bind serum albumin. For example, a column which comprises, e.g., Cibacron blue agarose (which has a high affinity for serum albumin) or anti-serum albumin antibodies can be used (see, e.g., FIGS. 2 and 4).

In yet another embodiment, a sample can be pre-fractionated by isolating proteins that have a specific characteristic, e.g. are glycosylated. For example, a blood serum sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Many types of affinity adsorbents exist which are suitable for pre-fractionating blood serum samples. An example of one other type of affinity chromatography available to pre-fractionate a sample is a single stranded DNA spin column. These columns bind proteins which are basic or positively charged. Bound proteins are then eluted from the column using eluants containing denaturants or high pH.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomolecules from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomolecules. In this embodiment, the sample is applied to a first adsorbent on the probe, which is subsequently washed with an eluant. Markers that do not bind to the first adsorbent are removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomolecules in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomolecules, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomolecules in a sample are separated by, e.g., isoelectric focusing, during which biomolecules in a sample are separated in a pH gradient until they reach a spot where their net charge is zero (i.e., isoelectric point). This first separation step results in one-dimensional array of biomolecules. The biomolecules in one dimensional array is further separated using a technique generally distinct from that used in the first separation step. For example, in the second dimension, biomolecules separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomolecules. Typically, two-dimensional gel electrophoresis can separate chemically different biomolecules in the molecular mass range from 1000-200,000 Da within complex mixtures.

Biomolecules in the two-dimensional array can be detected using any suitable methods known in the art. For example, biomolecules in a gel can be labeled or stained (e.g., Coomassie Blue or silver staining). If gel electrophoresis generates spots that correspond to the molecular weight of one or more markers of the invention, the spot is further analyzed by gas phase ion spectrometry. For example, spots can be excised from the gel and analyzed by gas phase ion spectrometry. Alternatively, the gel containing biomolecules can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI (e.g., using ProteinChip® array) as described in detail below.

Prior to gas phase ion spectrometry analysis, it may be desirable to cleave biomolecules in the spot into smaller fragments using cleaving reagents, such as proteases (e.g., trypsin). The digestion of biomolecules into small fragments provides a mass fingerprint of the biomolecules in the spot, which can be used to determine the identity of markers if desired.

In yet another embodiment, high performance liquid chromatography (HPLC) can be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers. For example, the spots can be analyzed using either MALDI or SELDI (e.g., using ProteinChip® array) as described in detail below.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass. spectrometry. In another example, biomolecules can be modified to improve detection resolution. For instance, neuraminidase can be used to remove terminal sialic acid residues from glycoproteins to improve binding to an anionic adsorbent (e.g., cationic exchange ProteinChip® arrays) and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

III. Capture of Markers

Biomarkers are preferably captured with capture reagents immobilized to a solid support, such as any biochip described herein, multiwell microtiter plate or a resin. In particular, the biomarkers of this invention are preferably captured on SELDI protein biochips. Capture can be on a chromatographic surface or a biospecific surface. Any of the SELDI protein biochips comprising reactive surfaces can be used to capture and detect the biomarkers of this invention. However, the biomarkers of this invention bind well to immobilized metal chelates. Thus, the IMAC-3 and IMAC 30 biochips, which nitriloacetic acid functionalities that adsorb transition metal ions, such as Cu++ and Ni++, by chelation, are the preferred SELDI biochips for capturing the biomarkers of this invention. SELDI biochips also can be derivatized with the antibodies that specifically capture the biomarkers, or they can be derivatized with capture reagents, such as protein A or protein G that bind immunoglobulins. Then the biomarkers can be captured in solution using specific antibodies and the captured markers isolated on chip through the capture reagent.

In general, a sample containing the biomarkers, such as serum, is placed on the active surface of a biochip for a sufficient time to allow binding. Then, unbound molecules are washed from the surface using a suitable eluant, such as phosphate buffered saline. In general, the more stringent the eluant, the more tightly the proteins must be bound to be retained after the wash. The retained protein biomarkers now can be detected by appropriate means.

IV. Detection and Characterrization of Markers

A. Spectrometry

Analytes captured on the surface of a protein biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention. Preferably, a laser desorption time-of-flight mass spectrometer is used in embodiments of the invention.

Matrix-assisted laser desorption/ionization mass spectrometry, or MALDI-MS, is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the biomarkers of this invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as proteins, in complex mixtures and is a preferred method of the present invention. SELDI is a method of mass spectrometry in which biomolecules, such as proteins, are captured on the surface of a protein biochip using capture reagents that are bound there. Typically, non-bound molecules are washed from the probe surface before interrogation. SELDI technology is available from Ciphergen Biosystems, Inc., Fremont Calif. as part of the ProteinChip® System. ProteinChip® arrays are particularly adapted for use in SELDI. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichester, 2000.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers. Preferably, markers captured on a protein biochip are detected using a laser desorption time-of-flight mass spectrometer, as described herein.

In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

In another embodiment, an ion mobility spectrometer can be used to detect markers. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

In yet another embodiment, a total ion current measuring device can be used to detect and characterize markers. This device can be used when the substrate has a only a single type of marker. When a single type of marker is on the substrate, the total current generated from the ionized marker reflects the quantity and other characteristics of the marker. The total ion current produced by the marker can then be compared to a control (e.g., a total ion current of a known compound). The quantity or other characteristics of the marker can then be determined.

B. Immunoassay

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

To prepare an antibody that specifically binds to a marker, purified markers or their nucleic acid sequences can be used. Nucleic acid and amino acid sequences for markers can be obtained by further characterization of these markers. For example, each marker can be peptide mapped with a number of enzymes (e.g., trypsin, V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 3rd Ed. (Sambrook et al., Cold Spring Harbor Laboratory, NY 2001).

Using the purified markers or their nucleic acid sequences, antibodies that specifically bind to a marker can be prepared using any suitable methods known in the art. See, e.g., Coligan, Current Protocols in Imnunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of e.g., a microtiter plate, a stick, a bead, or a microbead. Antibodies can also be attached to a probe substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

V. Data Analysis

Data generation in mass spectrometry begins with the detection of ions by an ion detector. A typical laser desorption mass spectrometer can employ a nitrogen laser at 337.1 nm. A useful pulse width is about 4 nanoseconds. Generally, power output of about 1-25 J is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-MIZ transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes that are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are in U.S. Provisional Patent Application Nos. 60/249,835, filed on Nov. 16, 2000, and 60/254,746, filed on Dec. 11, 2000, and U.S. Non-Provisional patent application Ser. Nos. 09/999,081, filed Nov. 15, 2001, and Ser. No. 10/084,587, filed on Feb. 25, 2002. All of these U.S. Provisional and Non Provisional patent applications are herein incorporated by reference in their entirety for all purposes.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., women in whom human cancer is undetectable).

V. Determination of Subject Status

Any biomarker, individually, is useful in aiding in the determination of breast cancer status. First, the selected biomarker is measured in a subject sample using the methods described herein, e.g., capture on a SELDI IMAC Ni biochip followed by detection by mass spectrometry. Then, the measurement is then compared with a diagnostic amount or control that distinguishes a breast cancer status from a non-cancer status. The diagnostic amount will reflect the information herein that a particular biomarker is up-regulated or down-regulated in a cancer status compared with a non-cancer status. As is well understood in the art, the particular diagnostic amount used can be adjusted to increase sensitivity or specificity of the diagnostic assay depending on the preference of the diagnostician. The test amount as compared with the diagnostic amount thus indicates breast cancer status.

While individual biomarkers are usefull diagnostic markers, it has been found that a combination of biomarkers provides greater predictive value than single markers alone. More particularly, Markers BC1, BC2 and BC3 are the most highly discriminatory biomarkers, used either alone or in combination.

In order to use the biomarkers in combination, a logistical regression algorithm is useful. The UMSA algorithm is particularly useful to generate a diagnostic algorithm from test data. This algorithm is disclosed in Z. Zhang et al., Applying classification separability analysis to microaary data. In: Lin S M, Johnson K F, eds. Methods of Microarray data analysis: papers from CAMDA '00. Boston: Kluwer Academic Publishers, 2001:125-136; and Z. Zhang et al., Fishing Expedition—a Supervised Approach to Extract Patterns from a Compendium of Expression Profiles. In Lin S M, Johnson, K F, eds. Microarray Data Analysis II: Papers from CAMDA '01. Boston: Kluwer Academic Publishers, 2002.

The learning algorithm will generate a multivariate classification (diagnostic) algorithm tuned to the particular specificity and sensitivity desired by the operator. The classification algorithm can then be used to determine breast cancer status. The method also involves measuring the selected biomarkers in a subject sample (e.g., Marker BC 1, BC2 and BC3). These measurements are submitted to the classification algorithm. The classification algorithm generates an indicator score that indicates breast cancer status.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

### General Comments

In the following Examples, the following Materials and Methods were used.

Samples.

Retrospective serum samples were obtained from the Johns Hopkins Clinical Chemistry serum banks, according to the approved protocol by the Johns Hopkins Joint Committee on Clinical Investigation. A total of 169 specimens were included in this study. The cancer group consisted 103 serum samples from breast cancer patients at different clinical stages: Stage 0 (n=4), Stage I (n=38), Stage II (n=37) and Stage III (n=24). Diagnoses were pathologically confirmed and specimens were obtained prior to treatment. Age information was not available on six of these patients. The median age of the remaining 96 patients was 56 years, ranging from 34 to 87 years. The non-cancer control group included serum from 25 with benign breast diseases (BN) and 41 healthy women (HC). Exact age information was not available from 21 healthy women. The median age of the remaining 20 healthy women was 45 years, ranging from 39 to 57 years. The median age of the benign condition group was 48 years with range between 21 and 78 years. All samples were stored at −80° C. until use.

ProteinChip Analysis.

To 20 μl of each serum sample, 30 μl of 8M urea, 1% CHAPS in PBS, PH 7.4 was added. The mixture was vortexed at 4° C. for 15 minutes and diluted 1:40 in PBS. Immobilized metal affinity capture chips (IMAC3) were activated with 50 mM NiSO₄ according to manufacturer's instructions (Ciphergen Biosystems, Inc., CA). 50 μl of diluted samples were applied to each spot on the ProteinChip array by using a 96 well bioprocessor (Ciphergen Biosystems, Inc., CA). After binding at room temperature for 60 minutes on a platform shaker, the array was washed twice with 100 μl of PBS for 5 minutes followed by two quick rinses with 100 μl of dH₂O. After air-drying, 0.5 μl of saturated sinapinic acid (SPA) prepared in 50% acetonitrile, 0.5% trifluoroacetic acid was applied twice to each spot. Proteins bound to the chelated metal (through histidine, tryptophan, cysteine or phosphorylated amino acids) were detected on a PBS-II mass reader. Data was collected by averaging 80 laser shots with an intensity of 240 and a detector sensitivity of 8. Reproducibility was estimated using two representative serum samples, one from the healthy controls and one from the cancer patients. Each serum sample was spotted on all 8 bait surfaces of one IMAC-Ni chip in each of the two bioprocessors. Coefficience of variance was estimated for the selected mass peaks.

Bioinformatics and Biostatistics.

Qualified mass peaks (S/N>5, cluster mass window at 0.3%) with M/Z between 2K and 150K were selected and the peak intensities were normalized to the total ion current using ProteinChip Software 3.0 (Ciphergen Biosystems, Inc., CA). Further preprocessing steps included logarithmic transformation applied to the peak intensity data in order to obtain a more consistent level of data variance across the entire range of spectrum of interest (M/Z 2 kD -150 kD).

The software package ProPeak (3Z Informatics, SC) was used to compute and rank the contribution of each individual peak towards the optimal separation of two diagnostic groups. ProPeak implements the linear version of the Unified Maximum Separability Analysis (UMSA) algorithm that was first reported for use in microarray data analysis. Z. Zhang et al., Applying Classification Separability Analysis to Microarray Data, in Proc. of Critical Assessment of Techniques for Microarray Data Analysis (CAMDA'00), Kluwer Academic Publishers, 2001. The key feature of the UIMSA algorithm is the incorporation of data distribution information into a structural risk minimization-learning algorithm (Vapnik VN, Statistical Learning Theory, John Wiley & Sons, Inc., New York, 199814) to identify a direction along which the two classes of data are best separated. This direction is represented as a linear combination (weighted sum) of the original variables. The weight assigned to each variable in this combination measures the contribution of the variable towards the separation of the two classes of data.

ProPeak offers three UMSA based analytical modules. The first is a Component Analysis module, which projects each specimen as an individual point onto a three-dimensional component space. The components (axes) are liner combinations of the original spectrum peak intensities. The axes correspond to directions along which two pre-specified groups of data achieve maximum separability. The separation between the two groups of data can be inspected in an interactive 3D display. The second module is Stepwise Selection, which uses a backward stepwise selection process to apply UMSA to compute a significance score for individual peaks and rank them according to their collective contribution towards the maximal separation of the two pre-specified groups of data. A positive or negative score indicates a relatively elevated or decreased expression level of the corresponding mass peak for the diseased group whereas the absolute value of the score represents its relative importance towards data separation. To avoid selecting peaks based on only unrelated artifacts in the data, the third module of ProPeak, BootStrap, uses a boot strap procedure to repeat UMSA for multiple runs each time randomly leaving out a fixed percentage of the samples from both groups. The median and mean ranks and the corresponding standard deviation are estimated for each peak. A potential biomarker should be a peak of top median and mean ranks and a minimum rank standard deviation. As a way to establish an objective selection criterion, the same bootstrap procedure was also applied to a random dataset that peak by peak simulate the distribution of the actual data. Results from the actual data are compared against the ones from the simulated data to establish a statistically appropriate cutoff value on rank standard deviation for selecting peaks with consistent performance.

### Example 1

### Identification of Biomarkers that Detect Breast Cancer at the Early Stages

In order to identify potential biomarkers that can detect breast cancer at early stages, protein profiles of specimens from stages 0-I breast cancer patients were compared against those of the non-cancer controls. The analysis was performed in multiple iterations using all three modules in ProPeak. Through this iterative process the original full spectrum was reduced to a small subset of mass peaks that had consistently demonstrated a high level of significance in the optimal separation between the two selected diagnostic groups.

Once a small panel of biomarkers was selected, their ability to detect breast cancer was independently tested using data from stages II and m cancer patients. Based on the entire data set, a composite index was derived using multivariate logistic regression. Descriptive statistics including p-values from two-sample t-tests were estimated. Receiver-operating-characteristic (ROC) curve analysis was then performed on the selected biomarkers and the composite index. Performance criteria such as sensitivities and specificities of the composite index were estimated using a bootstrap procedure. Efron B and Tibshirani R. Bootstrap Methods for Standard Errors, Confidence Intervals, and Other Measures of Statistical Accuracy. Statistical Science. 1986;1:54-75. In this procedure, the total patient data set was divided through random re-sampling into a training set to derive a composite index through logistic regression and a test set for computing sensitivities and specificities. This re-sampling process was repeated many times. The results from multiple runs were finally aggregated to form the bootstrap estimate of the sensitivities and specificities.

### Example 2

### Peak Detection and Data Preprocessing

Serum proteins retained on the IMAC-Ni²+ chips were analyzed on a PBS-II mass reader. A total of 147 qualified mass peaks (S/N>5, cluster mass window at 0.3%) with M/Z over 2 KD were selected. Peaks of M/Z less than 2 KD are excluded to eliminate interference from the matrix. Mass accuracy of 0.1% was achieved by external calibration using All In 1 Protein Standard (Ciphergen Biosystems, Inc., CA). A representative spectrum obtained from such analysis is shown in FIG. 2. Logarithmic transformation was applied to the peak intensity values. The plots in FIG. 3 illustrate the effect of variance reduction and equalization through logarithmic transformation.

### Example 3

### Biomarker Selection Based on Early-Stage Cancer and Non-Cancer Controls

To identify biomarkers with potential for early detection of breast cancer, UMSA was performed using early-stage cancer as the positive group (Stage 0-I n=42) and the non-cancer controls (HC+BN, n=66) as the negative group. Separability between the two groups was first tested using UMSA derived liner combination of all 147 mass peaks. The early-stage cancer was separable from the non-cancer group when the entire protein profiles were compared. FIG. 4A plots the early-stage cancer (lighter) versus non-cancer (darker) in the UMSA component 3D space.

To select biomarkers that consistently perform well, UMSA were applied repeatedly for a total of 100 runs each with 30% leave out rate using the ProPeak BootStrap module. The same procedure was also applied to a simulated random data set. The minimal standard deviation derived from the simulated data was 7. In the experimental data, 15 mass peaks had standard deviation less then this value. This subset of mass peaks was selected as candidate biomarkers for further analysis. Their mean ranks and the corresponding standard deviations are plotted in FIG. 4.

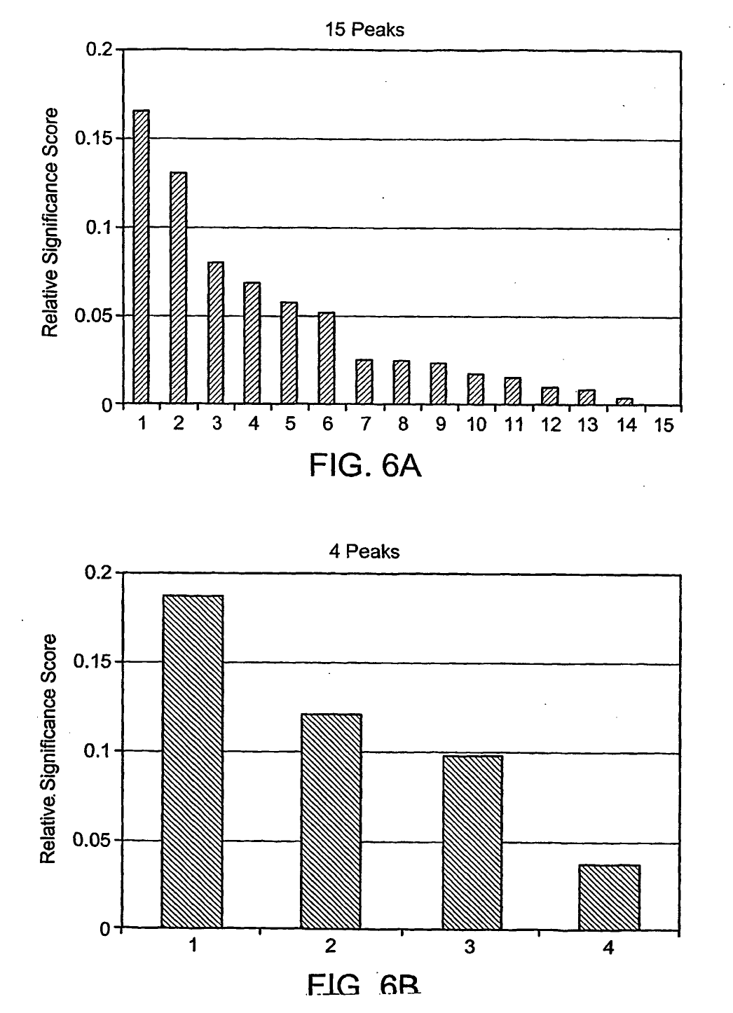

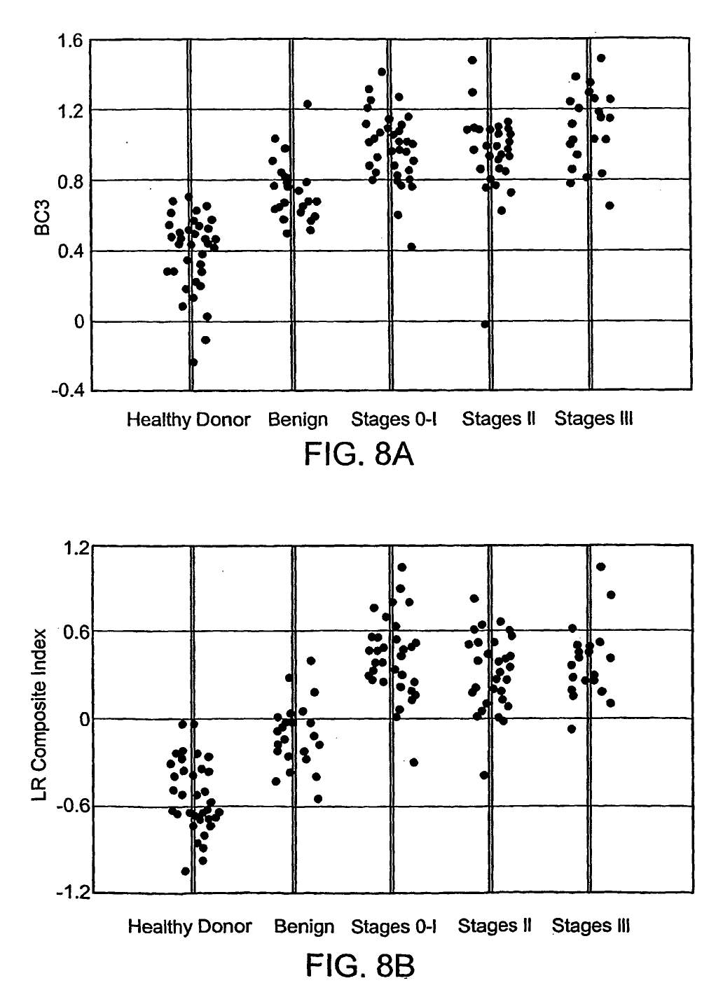

To further rank the peaks in this reduced set of candidate biomarkers, the Stepwise Selection module of ProPeak was applied. The absolute value of the relative significance scores of the 15 peaks (see Table 4) are plotted in descending order in FIG. 8A, which shows that the majority of separability between the two groups of data was contributed by the first six peaks. Among these six peaks, four are unique. The other two were identified as doubly charged forms of the two of the unique peaks using ProteinChip Software 3.0. The recognition of both the doubly charged and the singly charged forms of the peaks suggests their importance in discriminating the selected two diagnostic groups. Taking away the doubly charged forms, the four unique peaks were recombined and evaluated using Stepwise Selection again. The recalculated relative significance scores are plotted in FIG. 6B. The top-scored three peaks, designated BC1, BC2, and BC3, were fmally selected as the potential biomarkers for detection of breast cancer. BC1 appeared down regulated (scored negative) while BC2 and BC3 appeared up regulated (scored positive). A 3D-plot of stages 0-I breast cancer versus the non-cancer controls using these three biomarkers is shown in FIG. 4B.

### Example 4

### Evaluation of the Selected Biomarkers

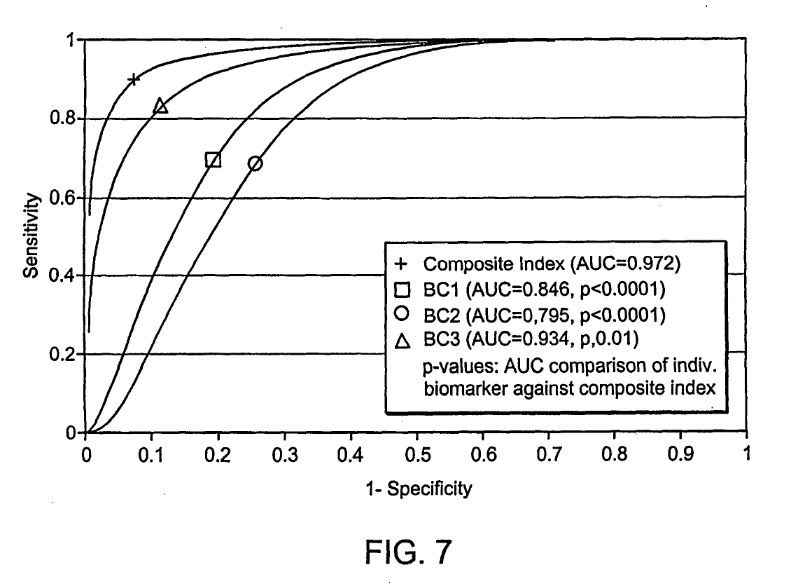

The descriptive statistics of these three biomarkers are listed in Table 1. FIG. 7 shows results from the ROC analysis. Among the three biomarkers, BC3 demonstrated the most individual diagnostic power. Its distributions over the diagnostic groups including clinical stages of cancer patients are plotted in FIG. 8A. The sensitivities and specificities of using BC3 alone at a cutoff value of 0.8 to differentiate the diagnostic groups are listed in Table 2A.

The estimated CV of the log transformed peak intensity was 6% for BC1, 7% for BC2, and 13% for BC3 (data not shown). Among the three biomarkers, BC3 had the largest CV of 13%. In comparison, the mean value of BC3 in the cancer patients was almost 90% above that in the non-cancer controls (calculated based on data in Table 1).

TABLE 1
Descriptive statistics of BC1, BC2, BC3, and the logistic regression
derived composite index. Differences between non-cancer controls
and stages 0-I, and between non-cancer controls and stages
II-III, are both statistically significant (p < 0.000001) for all three
biomarkers and the composite index.

| | Non-cancer | | Breast Cancer | | Breast Cancer | |
|---|---|---|---|---|---|---|
| | Controls | | Patients Stages | | Patients Stages | |
| | (n = 66) | | 0-I (n = 42) | | II-III (n = 61) | |
| | Mean | Stdev | Mean | Stdev | Mean | Stdev |
| BC1 | 0.302 | 0.312 | −0.118 | 0.244 | −0.081 | 0.258 |
| BC2 | 0.981 | 0.358 | 1.411 | 0.154 | 1.295 | 0.205 |
| BC3 | 0.526 | 0.252 | 0.993 | 0.193 | 1.003 | 0.234 |
| Comp. | −0.375 | 0.313 | 0.425 | 0.257 | 0.349 | 0.242 |
| Index | | | | | | |

### Example 5

### Combined Use of Three Selected Biomarkers

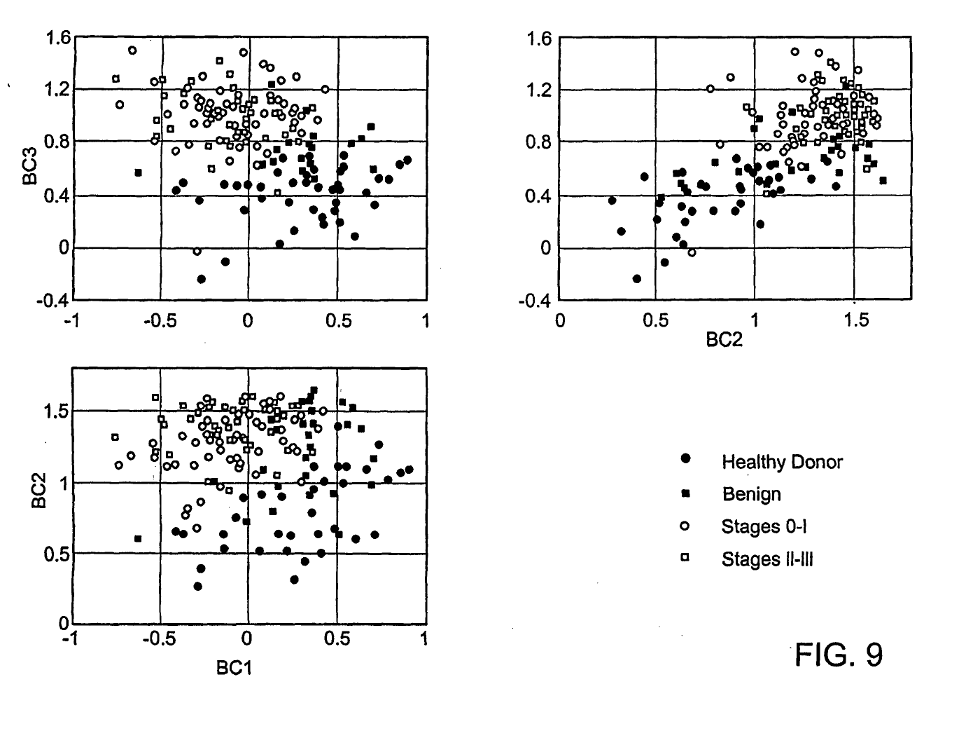

FIG. 9 compares the distribution of cancer patients at all clinical stages against non-cancer controls in all pair-wise biomarker combinations. Based on this observation, multivariate logistic regression was used to combine the three selected biomarkers to form a single-valued composite index. The descriptive statistics of this composite index are appended in Table 1. Its distributions over the various diagnostic group are plotted in FIG. 8B. ROC curve analysis of the composite index gave a much-improved AUC compared to the ones from individual biomarkers (FIG. 7).

Bootstrap cross-validation was used to estimate the diagnostic performance of the composite index (20 runs; in each run, 70% samples were randomly selected for composite index derivation and the remaining 30% for testing). The estimated sensitivities and specificities are listed in Table 2B.

The levels of the three potential biomarkers were also evaluated in relation to pT (tumor size) and pN (lymph node metastasis) categories. No significant correlation was observed.

TABLE 2A
Diagnostic performance of BC3.

| | | | | Breast Cancer Patients | | | |
|---|---|---|---|---|---|---|---|
| | Non-Cancer Controls | | | Stage | | | |
| Cutoff = 0.8 | HC | Benign | Subtotal | 0-I | II | III | Subtotal |
| Positive | 0 | 6 | 6 | 37 (88%) | 29 (78%) | 22 (92%) | 88 (85%) |
| Negative | 41 (100%) | 19 (76%) | 60 (91%) | 5 | 8 | 2 | 15 |
| Total | 41 | 25 | 66 | 42 | 37 | 24 | 103 |

TABLE 2B
BootStrap estimated diagnostic performance of logistic regression
derived composite index using BC1, BC2 and BC3 (20 runs, leave
out rate = 30%).

| LR at | Non-Cancer Controls | | | Breast Cancer Patients | | | |
|---|---|---|---|---|---|---|---|
| cutoff = | | Be- | | Stage | | | |
| 0 | HC | nign | Subtotal | 0-I | II | III | Subtotal |
| Positive | | | | 93% | 85% | 94% | 93% |
| | | | | | | | (85-100%) |
| Neg- | 100% | 85% | 91% | | | | |
| ative | | | (82-100%) | | | | |

### Example 6

### Detecting Breast Carcinoma In Situ by Serum Proteomic Analysis using ProteinChip® Arrays and SELDI-Mass Spectrometry

The protein profiles of 169 serum samples of women with and without breast cancer were analyzed, and a panel of three proteins (8.9 KD, 8.1 KD, 4.3 KD) were identified, that in combined use can detect breast cancer with high sensitivity (Stage 0-III, 93%) and specificity (Healthy Control+Benign, 91%). Among the three markers, the 8.9KD protein performed the best. A sensitivity of 85%, and a specificity of 91% were achieved.

Ductal and Lobular Carcinoma In Situ (DCIS and LCIS) are the earliest forms (Stage 0) of non-invasive breast cancer. Nearly 100% of women diagnosed at this early stage of breast cancer can be cured. To validate these markers for early detection of breast cancer, the performance of the 3 previously identified biomarkers were evaluated using sera collected by a collaborating institution. The sample cohort consisted of 17 women with DCIS, 1 with LCIS, 8 with benign breast diseases, and 40 age-matched apparently healthy controls (45-65 years). Protein profiles were generated in triplicates using IMAC-Ni (Immobilized Metal Affinity Capture) ProteinChip arrays under the same experimental conditions as described supra. Log relative intensities of each of the three proteins were compared between different diagnostic groups using two-sample t-test. The expression patterns of two (8.9 KD and 8.1KD) of the three markers were consistent with previous results. The p-values and the areas under the ROC curves of these two biomarkers are summarized in Table 3.

TABLE 3
Summary of Statistical Analysis

| | Two-sample t-test | | Area under the | | Diagnostic performance | | |
|---|---|---|---|---|---|---|---|
| | p-value | | ROC-curve | | Sensitivity | Specificity | Specificity |
| | DCIS/HC | DCIS/HC + BN | DCIS/HC | DCIS/HC + BN | (DCIS) | (HC) | (HC + BN) |
| 8.9 KD | 0.000059 | 0.000072 | 0.80 | 0.76 | 72% (13/18) | 65% (26/40) | 63% (30/48) |
| 8.1 KD | 0.0180 | 0.0194 | 0.76 | 0.71 | 61% (11/18) | 75% (30/40) | 75% (36/48) |

DCIS, Ductal Carcinoma In Situ;
LCIS, Lobular Carcinoma In Situ;
HC, Healthy Control;
BN, Benign

The following specific references also are incorporated by reference herein.
1. Jemal A, Thomas A, Murray T, Thun M. Cancer statistics, 2002. CA Cancer J Clin. 2002; 52:23-47.
2. National Cancer Institute. Cancer Net PDQ Cancer Information Summaries. Monographs on “Screening for breast cancer.” http://cancer net.nci.nih.gov/pdq.html (Updated January 2001).
3. Antman K, Shea S. Screening mammography under age 50. JAMA. 1999; 281:1470-2.
4. Chan D W, Beveridge R A, Muss H, Fritsche H A, Hortobagyi G, Theriault R, et al. Use of Truquant BR Radioimmunoassay for early detection of breast cancer recurrence in patients with stage II and stage III disease, J Clin. Oncology. 1997; 15:2322-2328.
5. Karas M, Hillenkamp F. Laser desorption ionization of proteins with molecular masses exceeding 10,000 daltons. Anal Chem. 1988; 60:2299-2301.
6. Hutchens T W, Yip T T. New desorption strategies for the mass spectrometric analysis of micromolecules. Rapid Commun. Mass Spectrom. 1993; 7:576-80.
7. Merchant M, Weinberger S R. Recent advancements in surface-enhanced laser desorption/ionization-time of flight-mass spectrometry. Electrophoresis. 2000; 21:1164-67.
8. Wright Jr G L, Cazares L H, Leung S-M, Nasim S, Adam B-L, Yip T-T, et al. ProteinChip® surface enhanced laser desorption/ionization (SELDI) mass spectrometry: a novel protein biochip technology for detection of prostate cancer biomarkers in complex protein mixtures. Prostate Cancer Prostate Dis. 1999; 2:264-76.
9. Hlavaty J J, Partin A W, Kusinitz F, Shue M J, Stieg M, Bennett K, Briggman J V. Mass spectroscopy as a discovery tool for identifying serum markers for prostate cancer. Clin. Chem. [Abstract]. 2001; 47:1924-26.
10. Paweletz C P, Trock B, Pennanen M, Tsangaris T, Magnant C, Liotta L A, et al. Proteomic patterns of nipple aspirate fluids obtained by SELDI-TOF: potential for new biomarkers to aid in the diagnosis of breast cancer. Dis Markers. 2001;17:301-7.
11. Vlahou A, Schellhammer P F, Medrinos S, Patel K, Kondylis F I, Gong L, et al. Development of a novel proteomic approach for the detection of transitional cell carcinoma of the bladder in urine. Am J Pathol. 2001; 158:1491-502.
12. Patricoin E F III, Ardekani A M, Hitt B A, Levine P J, Fusaro V A, Steinberg S M, et al.
Use of proteomic patterns in serum to identify ovarian cancer. The Lancet. 2002; 359:572-577.
13. Zhang Z, Page G, Zhang H. Applying Classification Separability Analysis to Microarray Data, in Proc. of Critical Assessment of Techniques for Microarray Data Analysis (CAMDA'00), Kluwer Academic Publishers, 2001.
14. Vapnik V N, Statistical Learning Theory, John Wiley & Sons, Inc., New York, 1998.
15. Efron B and Tibshirani R. Bootstrap Methods for Standard Errors, Confidence Intervals, and Other Measures of Statistical Accuracy. Statistical Science. 1986; 1:54-75.

The invention has been described in detail with reference to particular embodiments thereof. However, it will be appreciated that those skilled in the art, upon consideration of this disclosure, may make modifications within the spirit and scope of the invention.

1. A method of qualifying breast cancer in a subject comprising:
(a) measuring at least one biomarker in a sample from a subject, and
(b) correlating the measurement with breast cancer status,
wherein the biomarker is selected from the group consisting of:
Marker I (BC1)
Marker II (BC2)
Marker III (BC3)
Marker IV
Marker V
Marker VI
Marker VII
Marker VIII
Marker IX
Marker X
Marker XI
Marker XII
Marker XIII
Marker XIV,
and combinations thereof.

2. The method of claim 1 further comprising:
(c) managing subject treatment based on the status.

3. The method of claim 2 wherein managing subject treatment is selected from ordering more rests, performing surgery, and taking no further action.

4. The method of claim 2 further comprising:
(d) measuring the at least one biomarker after subject management.

5. The method of claim 1 wherein the breast cancer status is selected from the group consisting of the subject's risk of cancer, the presence or absence of disease, the stage of disease, and the effectiveness of treatment.

6. The method of claim 1 wherein measuring comprises detecting by mass spectrometry.

7. The method of claim 1 wherein at least one biomarker is selected from Marker I (BC1), Marker II (BC2), and Marker III (BC3).

8. The method of claim 1 further comprising measuring a known breast cancer biomarker in a sample from the subject and correlating measurement of the known biomarker and the measurement of any one or more of Markers I through XIV with breast cancer status.

9. The method of claim 8 wherein the known biomarker is selected from CA 15.3 or CA 27.29.

10. The method of claim 1 comprising measuring Marker I (BC1), Marker II (BC2), and Marker III (BC3).

11. The method of claim 10 further comprising measuring a known breast cancer biomarker in a sample from the subject and correlating measurement of the known biomarker and the measurement of any one or more of Markers I through XIV with breast cancer status.

12. The method of claim 11 wherein the known biomarker is selected from CA 15.3 or CA 27.29.

13. The method of claim 1 wherein measuring comprises:
(a) providing a subject sample of blood or a blood derivative;
(b) capturing one or more of Markers I through XIV from the sample on a surface of a substrate comprising capture reagents that bind the protein biomarkers.

14. The method of claim 13 wherein the substrate is a SELDI probe comprising an IMAC Ni surface and wherein the protein biomarkers are detected by SELDI.

15. The method of claim 13 wherein the substrate is a SELDI probe comprising biospecific affinity reagents that bind one or more of Markers I through XIV and wherein the protein biomarkers are detected by SELDI.

16. The method of claim 13 wherein the substrate is a microtiter plate comprising biospecific affinity reagents that bind one or more of Markers I through XV and the protein biomarkers are detected by immunoassay.

17. The method of claim 1 wherein measuring is selected from detecting the presence or absence of the biomarkers(s), quantifying the amount of marker(s), and qualifying the type of biomarker.

18. The method of claim 1 wherein at least one biomarker is measured using a biochip array.

19. The method of claim 18 wherein the biochip array is a protein chip array.

20. The method of claim 18 wherein the biochip array is a nucleic acid array.

21. The method of claim 18 wherein at least one biomarker is immobilized on the biochip array.

22. The method of claim 1 wherein the protein biomarkers are measured by SELDI.

23. The method of claim 1 wherein the protein biomarkers are measured by immunoassay.

24. The method of claim 1 wherein the correlating is performed by a software classification algorithm.

25. The method of claim 1 wherein the sample is selected from blood, serum and plasma.

26. A method comprising:
(a) measuring a plurality of biomarkers in a sample from the subject, wherein the plurality of biomarkers is selected from the group consisting of:
Marker I (BC1)
Marker II (BC2)
Marker III (BC3)
Marker IV
Marker V
Marker VI
Marker VII
Marker VIII
Marker IX
Marker X
Marker XI
Marker XII
Marker XIII, and
Marker XIV.

27. The method of claim 26 wherein the plurality includes Marker I (BC1), Marker II (BC2), and Marker III (BC3).

28. The method of claim 26 further comprising measuring a known biomarker.

29. The method of claim 26 wherein the known biomarker is selected from CA 15.3 or CA 27.29.

30. The method of claim 26 wherein the protein biomarkers are detected by SELDI or immunoassay.

31. The method of claim 26 wherein the sample is selected from blood, serum and plasma.

32. A method comprising:
(a) measuring at least one biomarker in a sample from a subject, wherein the biomarker is selected from the group consisting of:
Marker I (BC1)
Marker II (BC2)
Marker III (BC3)
Marker IV
Marker V
Marker VI
Marker VII
Marker VIII
Marker IX
Marker X
Marker XI
Marker XII
Marker XIII, and
Marker XIV,
and combinations thereof.

33. The method of claim 32 further comprising measuring a known biomarker.

34. The method of claim 33 wherein the known biomarker is selected from CA 15.3 or CA 27.29.

35. The method of claim 32 wherein the protein biomarkers are detected by SELDI or immunoassay.

36. The method of claim 32 wherein the sample is selected from blood, serum and plasma.

37. A kit comprising:
(a) a capture reagent that binds a biomarker selected from the group consisting of:
Marker I (BC1)
Marker II (BC2)
Marker III (BC3)
Marker IV
Marker V
Marker VI
Marker VII
Marker VIII
Marker IX
Marker X
Marker XI
Marker XII
Marker XIII, and
Marker XIV,
and combinations thereof; and
(b) a container comprising at least one of the biomarkers.

38. The kit of claim 37 wherein the capture reagent binds a plurality of the biomarkers.

39. The kit of claim 37 wherein the capture reagent is a SELDI probe.

40. The kit of claim 37 further comprising a capture reagent that binds CA 15.3 or CA 27.29.

41. The kit of claim 37 further comprising a second capture reagent that binds one of the biomarkers that the first capture reagent does not bind.

42. A kit comprising:
(a) a first capture reagent that binds at least one biomarker selected from the group consisting:
Marker I (BC1)
Marker II (BC2)
Marker III (BC3)
Marker IV
Marker V
Marker VI
Marker VII
Marker VIII
Marker IX
Marker X
Marker XI
Marker XII
Marker XIII, and
Marker XIV; and
(b) a second capture reagent that binds at least one of the biomarkers that is not bound by the first capture reagent.

43. The kit of claim 42 wherein the at least one capture reagent is an antibody.

44. The kit of claim 42 further comprising an MS probe to which at least one capture reagent is attached or is attachable.

45. The kit of claim 42 wherein the capture reagent is an immobilized metal chelate (IMAC).

46. The kit of claim 42 further comprising a wash solution that selectively allows retention of the bound biomarker to the capture reagent as compared with other biomarkers after washing.

47. A kit comprising:
(a) a first capture reagent that binds at least one biomarker selected from the group consisting of:
Marker I (BC1)
Marker II (BC2)
Marker III (BC3)
Marker IV
Marker V
Marker VI
Marker VII
Marker VII
Marker IX
Marker X
Marker XI
Marker XII
Marker XIII, and
Marker XIV;
(b) instructions for using the capture reagent to detect the biomarker.

48. The kit of claim 47 wherein the capture reagent is an antibody.

49. The kit of claim 47 further comprising an MS probe to which the capture reagent is attached or is attachable.

50. The kit of claim 47 wherein the capture reagent is an immobilized metal chelate (IMAC).

51. The kit of claim 47 further comprising a wash solution that selectively allows retention of the bound biomarker to the capture reagent as compared with other biomarkers after washing.

52. The kit of claim 47 further comprising written instructions for use of the kit for detection of cancer.

53. The kit of claim 52 wherein the instructions provide for contacting a test sample with the capture agent and detecting one or more biomarkers retained by the capture agent.

54. An article manufacture comprising:
(a) at least one capture reagent that binds to at least two biomarkers selected from the group consisting of:
Marker I (BC1)
Marker II (BC2)
Marker III (BC3)
Marker IV
Marker V
Marker VI
Marker VII
Marker VIII
Marker IX
Marker X
Marker XI
Marker XII
Marker XIII, and
Marker XIV.

55. The article of manufacture of claim 54 further comprising a capture reagent that binds to a known biomarker.

56. The article of manufacture of claim 55 wherein the known biomarker is CA 15.3 or CA 27.29.

57. A system comprising:
(a) a plurality of capture reagents each of which has bound to it a different biomarker selected from the group consisting of:
Marker I (BC1)
Marker II (BC2)
Marker III (BC3)
Marker IV
Marker V
Marker VI
Marker VII
Marker VIII
Marker IX
Marker X
Marker XI
Marker XII
Marker XIII, and
Marker XIV.
